(19) United States
(12) Patent Application Publication
(10) Pub. No.: US 2023/0263772 A1
(43) Pub. Date: Aug. 24, 2023

(54) COMPOSITIONS AND METHODS FOR TREATING PAIN AND/OR INFLAMMATION

(71) Applicant: Neumentum, Inc., Palo Alto, CA (US)

(72) Inventors: Joseph V. Pergolizzi, Naples, FL (US); Robert B. Raffa, Tucson, AZ (US); Ilona Steigerwald, Aachen (DE)

(21) Appl. No.: 18/106,084

(22) Filed: Feb. 6, 2023

Related U.S. Application Data
(63) Continuation of application No. 17/486,280, filed on Sep. 27, 2021, now abandoned.
Application No. 17/486,280 is a continuation of application No. PCT/US21/27228, filed on Apr. 14, 2021.
(60) Provisional application No. 63/010,331, filed on Apr. 15, 2020.

(51) Int. Cl. A61K 31/415; A61P 23/00; A61K 45/06; A61K 47/10; A61K 47/18

(52) U.S. Cl. CPC A61K 31/415; A61P 23/00; A61K 45/06; A61K 47/10; A61K 47/18

(57) ABSTRACT

Disclosed in certain embodiments is a method for treating chronic pain with a hetero-substituted acetanilide compound that modulates the adenosine receptor. Disclosed in certain embodiments is a method for treating neuropathic pain (e.g., acute or chronic) with a hetero-substituted acetanilide compound that modulates the adenosine A3 receptor. Disclosed in certain embodiments is a method for treating inflammation (e.g., local inflammation or systemic inflammation), an inflammatory response, or an inflammatory condition with a hetero-substituted acetanilide compound.

### FIELD OF THE INVENTION

The present disclosure is directed to pharmaceutical compositions and methods directed to the treatment of pain, such as chronic pain (e.g., chronic neuropathic pain) or acute neuropathic pain. In certain embodiments, the present disclosure is directed to pharmaceutical compositions and methods directed to the treatment of inflammation.

### BACKGROUND OF THE INVENTION

Pain management includes the use of compositions and methods to treat pain from surgery, injury, or illness. Pain can cause changes in a patient's physical and emotional health, such as depression and sleep problems. Pain management may help the patient to rest, heal, and return to daily activities with no or minimal disruption. Pain management can also help increase a patient's appetite, sleep, and energy, and improve mood and relationships. More active analgesic compositions are in constant demand because they offer the attractive possibility of relieving pain with a reduced side effect profile. The side effect profile can be due to the characteristics of the compound such as the mechanism of action or increased potency which can result in decreased dosages, thereby diminishing the expected side effects and toxicity that would otherwise result from available dosage forms.

There is a particular need in the art for the development and availability of more active analgesic combinations because they offer the attractive possibility of relieving pain through the actions of multiple mechanism of actions and offer the possibility of reduced dosages and a more acceptable side effect profile.

### OBJECTS AND SUMMARY OF THE INVENTION

It is an object of certain embodiments of the present invention to provide pharmaceutical compositions and methods for the treatment of pain, such as chronic pain.

It is an object of certain embodiments of the present invention to provide pharmaceutical compositions and methods for the treatment of inflammation.

The above objects and others are met by the present invention which in certain embodiments is directed to methods for the treatment of chronic pain by administering to a patient in need thereof a therapeutically effective amount of a hetero-substituted acetanilide compound.

In certain embodiments, the chronic pain includes neuropathic pain, nociceptive pain, or a combination thereof.

In certain embodiments, the present invention is directed to methods for the treatment of neuropathic pain by administering to a patient in need thereof a therapeutically effective amount of a hetero-substituted acetanilide compound. The neuropathic pain can be acute or chronic in duration.

In certain embodiments, the hetero-substituted acetanilide compound modulates the adenosine A3 receptor.

In certain embodiments, the present invention is directed to methods for the treatment of inflammation by administering to a patient in need thereof a therapeutically effective amount of a hetero-substituted acetanilide compound. The inflammation can be a local inflammatory response or a systemic inflammation.

In certain embodiments, the hetero-substituted acetanilide compound is selected from Formula (I) as disclosed herein, Formula (II) as disclosed herein or a combination thereof.

In certain embodiments, Formula (I) is

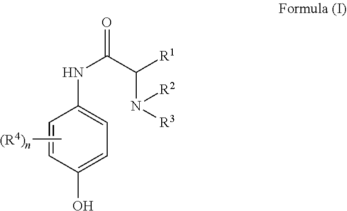

wherein R¹ and R² taken together with the atoms to which they are attached form a radical selected from the group consisting of a 5 to 10 membered cycloheteroalkanyl, 5 to 10 membered cycloheteroalkenyl, a 5 to 10 membered heteroaryl, and 5 to 10 membered cycloheteroalkyl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;
R³ is hydrogen or C₁₋₆alkanyl;
R⁴ is a substituent selected from the group consisting of hydrogen, C₁₋₆alkanyl. C₁₋₆alkanyloxy, fluorinated alkanyl, fluorinated alkanyloxy, halogen, hydroxyl, nitro, amino, C₁₋₆alkanylamino; C₁₋₆dialkanylamino and cyano;
n is an integer from 1 to 3; and
enantiomers, diastereomers, tautomers, solvates, or pharmaceutically acceptable salts thereof.

In certain embodiments, Formula (II) is

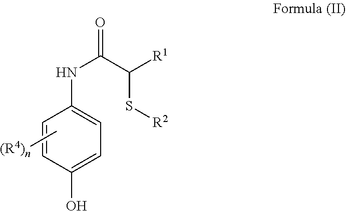

wherein R¹ is a substituent independently selected from the group consisting of hydrogen and C₁₋₆alkanyl;

R² is a substituent independently selected from the group consisting of hydrogen, C₁₋₆alkanyl and dioxo; or

R¹ and R² taken together with the atoms to which they are attached form a radical selected from the group consisting of a 5 to 10 membered cycloheteroalkanyl, 5 to 10 membered cycloheteroalkenyl, a 5 to 10 membered heteroaryl, and cycloheteroalkyl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo:

R⁴ is a substituent selected from the group consisting of hydrogen, C₁₋₆alkanyl, C₁₋₆alkanyloxy, fluorinated alkanyl, fluorinated alkanyloxy, halogen, hydroxyl, nitro, amino, C₁₋₆alkanylamino; C₁₋₆dialkanylamino and cyano;

n is an integer from 1 to 3; and

enantiomers, diastereomers, tautomers, solvates, or pharmaceutically acceptable salts thereof.

### BRIEF DESCRIPTION OF THE DRAWINGS

The above and other features of the present invention, their nature, and various advantages will become more apparent upon consideration of the following detailed description, taken in conjunction with the accompanying drawings, in which:

FIG. 1 illustrates the effect of a hetero-substituted acetanilide compound (300 mg/kg, oral), according to an embodiment, in Phase II of a rat formalin (1%) model as compared to a negative control (vehicle only) and to a positive control (Tramadol 50 mg/kg). The test and results shown in FIG. 1 are described in Example 1.

FIG. 2A illustrates the dose related effect of a hetero-substituted acetanilide compound (300 mg/kg and 600 mg/kg, orally), according to an embodiment, in a carrageenan-induced paw-edema model as compared to a control (vehicle only). The test and results shown in FIG. 2A are described in Example 2.

FIG. 2B illustrates the area under the curve (AUC) of the chart in FIG. 2A.

FIG. 3 illustrates Pain Intensity Difference (PID) of a hetero-substituted acetanilide compound

### DEFINITIONS

As used herein, the singular forms “a,” “an,” and “the” include plural references unless the context clearly indicates otherwise. Thus, for example, reference to “a drug” includes a single drug as well as a mixture of two or more different drugs and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number 10%, such that “about 10” would include from 9 to 11.

As used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

The term “subject” encompasses the definition of the term “patient” and does not exclude individuals who are otherwise healthy.

The terms “treatment of” and “treating” include the administration of the disclosed combination of drugs with the intent to lessen the severity of or prevent a condition and is not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

The terms “prevention of” and “preventing” include (1) inhibiting or avoiding the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “condition” or “conditions” refers to those medical conditions, such as pain and/or inflammation, that can be treated, mitigated or prevented by administration to a subject of an effective amount of the disclosed combination.

An “effective amount” refers to the amount of an active agent or combination of active agents that is sufficient to produce a beneficial or desired effect at a level that is readily detectable or observed by a method commonly used for detection and observation of such an effect. In some embodiments, such an effect results in a change of at least 10% from the value of a basal level where the active agent is not administered. In other embodiments, the change is at least 20%, 50%, 80%, or an even higher percentage from the basal level. As will be described below, the effective amount may vary from subject to subject, depending on age, general condition of the subject, the severity of the condition being treated, the particular active agent administered and the like. An appropriate “effective” amount in any individual case may be determined by one of ordinary skill in the art by reference to the pertinent texts and literature and/or by using routine experimentation.

As used herein, the terms “active agent” or “drug” or “active ingredient” refer to any material that is intended to produce a therapeutic, prophylactic, or other intended effect, whether or not approved by a government agency for that purpose.

“C_{a-b}” (where a and b are integers) refers to a radical containing from a to b carbon atoms inclusive. For example, C₁₋₃ denotes a radical containing 1, 2 or 3 carbon atoms

“Alkyl” refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl”, “alkenyl” and/or “alkynyl” is used, as defined below. In preferred embodiments, the alkyl groups are (C₁-C₆) alkyl, with (C₁-C₃) being particularly preferred.

“Alkanyl” refers to a saturated branched, straight-chain or cyclic monovalent hydrocarbon radical derived by the removal of one hydrogen atom from a single carbon atom of a parent alkane. Typical alkanyl groups include, but are not limited to, methanyl; ethanyl; propanyls such as propan-1-yl, propan-2-yl, cyclopropan-1-yl, etc.; butyanyls such as butan-1-yl, butan-2-yl, 2-methyl-propan-1-yl, 2-methyl-propan-2-yl, cyclobutan-1-yl, etc.; and the like. In preferred embodiments, the alkanyl groups are (C₁₋₈) alkanyl, with (C₁₋₃) being particularly preferred.

“Alkenyl” refers to an unsaturated branched, straight-chain or cyclic monovalent hydrocarbon radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” refers to an unsaturated branched, straight-chain or cyclic monovalent hydrocarbon radical having at least one carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Heteroalkyl” and “Heteroalkanyl” refer to alkyl or alkanyl radicals, respectively, in which one or more carbon atoms (and any necessary associated hydrogen atoms) are independently replaced with the same or different heteroatoms (including any necessary hydrogen or other atoms). Typical heteroatoms to replace the carbon atom(s) include, but are not limited to, N. P, O, S, Si, etc. Preferred heteroatoms are O, N and S. Thus, heteroalkanyl radicals can contain one or more of the same or different heteroatomic groups, including, by way of example and not limitation, epoxy (—O—), epidioxy (—O—O—), thioether (—S—), epidithio (—SS—), epoxythio (—O—S—), epoxyimino (—O—NR′—), imino (—NR′—), biimmino (—NR′—NR′—), azino (═N—N═), azo (—N═N—), azoxy (—N—O—N—), azimino (—NR′—N═N—), phosphano (—PH—), λ⁴-sulfano (—SH₂—), sulfonyl (—S(O)₂—), and the like, where each R′ is independently hydrogen or (C₁-C₆) alkyl.

“Parent Aromatic Ring System” refers to an unsaturated cyclic or polycyclic ring system having a conjugated π electron system. Specifically included within the definition of “parent aromatic ring system” are fused ring systems in which one or more rings are aromatic and one or more rings are saturated or unsaturated, such as, for example, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Aryl” refers to a monovalent aromatic hydrocarbon radical derived by the removal of one hydrogen atom from a single carbon atom of a parent aromatic ring system. Typical aryl groups include, but are not limited to, radicals derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅₋₂₀) aryl, with (C₅₋₁₀) being particularly preferred. Particularly preferred aryl groups are phenyl and naphthyl groups.

“Arylalkyl” refers to an acyclic alkyl group in which one of the hydrogen atoms bonded to a carbon atom, typically a terminal carbon atom, is replaced with an aryl radical Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C₆₋₂₆) arylalkyl, e.g., the alkanyl, alkenyl or alkynl moiety of the arylalkyl group is (C₁₋₆) and the aryl moiety is (C₅₋₂₀). In particularly preferred embodiments the arylalkyl group is (C₆₋₁₃), e.g., the alkanyl, alkenyl or alkynyl moiety of the arylalkyl group is (C₁₋₃) and the aryl moiety is (C₅₋₁₀). Even more preferred arylalkyl groups are phenylalkanyls.

“Alkanyloxy” refers to a saturated branched, straight-chain or cyclic monovalent hydrocarbon alcohol radical derived by the removal of the hydrogen atom from the hydroxide oxygen of the alcohol. Typical alkanyloxy groups include, but are not limited to, methanyl; ethanyloxy; propanyloxy groups such as propan-1-yloxy (CH₃CH₂CH₂O—), propan-2-yloxy ((CH₃)₂CHO—), cyclopropan-1-yloxy, etc.; butyanyloxy groups such as butan-1-yloxy, butan-2-yloxy, 2-methyl-propan-1-yloxy, 2-methyl-propan-2-yloxy, cyclobutan-1-yloxy, etc.; and the like. In preferred embodiments, the alkanyloxy groups are (C₁₋₈) alkanyloxy groups, with (C₁₋₃) being particularly preferred.

“Parent Heteroaromatic Ring System” refers to a parent aromatic ring system in which one carbon atom is replaced with a heteroatom. Heteratoms to replace the carbon atoms include N, O, and S. Specifically included within the definition of “parent heteroaromatic ring systems” are fused ring systems in which one or more rings are aromatic and one or more rings are saturated or unsaturated, such as, for example, arsindole, chromane, chromene, indole, indoline, xanthene, etc. Typical parent heteroaromatic ring systems include, but are not limited to, carbazole, imidazole, indazole, indole, indoline, indolizine, isoindole, isoindoline, isoquinoline, isothiazole, isoxazole, naphthyridine, oxadiazole, oxazole, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” refers to a monovalent heteroaromatic radical derived by the removal of one hydrogen atom from a single atom of a parent heteroaromatic ring system. Typical heteroaryl groups include, but are not limited to, radicals derived from carbazole, imidazole, indazole, indole, indoline, indolizine, isoindole, isoindoline, isoquinoline, isothiazole, isoxazole, naphthyridine, oxadiazole, oxazole, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-20 membered heteroaryl, with 5-10 membered heteroaryl being particularly preferred.

“Cycloheteroalkyl” refers to a saturated or unsaturated monocyclic or bicyclic alkyl radical in which one carbon atom is replaced with N, O or S. In certain specified embodiments the cycloheteroalkyl may contain up to four heteroatoms independently selected from N, O or S. Typical cycloheteroalkyl moieties include, but are not limited to, radicals derived from imidazolidine, morpholine, piperazine, piperidine, pyrazolidine, pyrrolidine, quinuclidine, and the like. In preferred embodiments, the cycloheteroalkyl is a 3-6 membered cycloheteroalkyl.

“Cycloheteroalkanyl” refers to a saturated monocyclic or bicyclic alkanyl radical in which one carbon atom is replaced with N, O or S. In certain specified embodiments the cycloheteroalkanyl may contain up to four heteroatoms independently selected from N, O or S. Typical cycloheteroalkanyl moieties include, but are not limited to, radicals derived from imidazolidine, morpholine, piperazine, piperidine, pyrazolidine, pyrrolidine, quinuclidine, and the like. In preferred embodiments, the cycloheteroalkanyl is a 3-6 membered cycloheteroalkanyl.

“Cycloheteroalkenyl” refers to a saturated monocyclic or bicyclic alkenyl radical in which one carbon atom is replaced with N, O or S. In certain specified embodiments the cycloheteroalkenyl may contain up to four heteroatoms independently selected from N, O or S. Typical cycloheteroalkenyl moieties include, but are not limited to, radicals derived from imidazoline, pyrazoline, pyrroline, indoline, pyran, and the like. In preferred embodiments, the cycloheteroalkanyl is a 3-6 membered cycloheteroalkanyl.

“Substituted” refers to a radical in which one or more hydrogen atoms are each independently replaced with the same or different substituent(s). Typical substituents include, but are not limited to, —X, —R, —O⁻,═O, —OR, —O—OR, —SR, —S⁻, ═S, —NRR,═NR, —CX₃, —CN, —OCN, —SCN, —NCO, —NCS, —NO, —NO₂, ═N₂, —N₃, —NHOH, —S(O)₂O⁻, —S(O)₂OH, —S(O)₂R, —P(O)(O⁻)₂, —P(O)(OH)₂, —C(O)R, —C(O)X, —C(S)R, —C(S)X, —C(O)OR, —C(O)O—, —C(S)OR, —C(O)SR, —C(S)SR, —C(O)NRR, —C(S)NRR and —C(NR)NRR, where each X is independently a halogen (preferably —F, —Cl or —Br) and each R is independently —H, alkyl, alkanyl, alkenyl, alkynyl, alkylidene, alkylidyne, aryl, arylalkyl, arylheteroalkyl, heteroaryl, heteroarylalkyl or heteroaryl-heteroalkyl, as defined herein. Preferred substituents include hydroxy, halogen, C₁₋₈alkyl. C₁₋₈ alkanyloxy, fluorinated alkanyloxy, fluorinated alkyl, C₁₋₈alkylthio, C₃₋₈cycloalkyl, C₃₋₈ cycloalkanyloxy, nitro, amino, C₁₋₈alkylamino, C₁₋₈dialkylamino, C₃₋₈cycloalkylamino, cyano, carboxy, C₁₋₇alkanyloxycarbonyl, C₁₋₇alkylcarbonyloxy, formyl, carbamoyl, phenyl, aroyl, carbamoyl, amidino, (C₁₋₈alkylamino)carbonyl, (arylamino)carbonyl and aryl(C₁₋₈ alkyl)carbonyl.

With reference to substituents, the term “independently” means that when more than one of such substituent is possible, such substituents may be the same or different from each other.

Throughout this disclosure, the terminal portion of the designated side chain is described first, followed by the adjacent functionality toward the point of attachment. Thus, for example, a “phenylC₁₋₆alkanylaminocarbonylC₁₋₆alkyl” substituent refers to a group of the formula

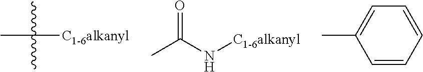

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

In structures wherein stereochemistry is not explicitly indicated, it is assumed that either stereochemistry is considered and both isomers claimed.

Any undefined valency on an atom of a structure shown in this application implicitly represents a hydrogen atom bonded to the atom.

An “isomer” of a first compound is a separate compound in which each molecule contains the same constituent atoms as the first compound, but where the configuration of those atoms in three dimensions differs.

The term “Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. In a specific embodiment, the term “pharmaceutically acceptable” means approved by a regulatory agency of the federal or a state government or listed in the U.S. Pharmacopeia or other generally recognized pharmacopeia for use in animals, and more particularly in humans.

The phrase “controlled release” as used herein refers to a formulation capable of releasing the active ingredient at a predetermined rate such that therapeutically beneficial levels are kept over an extended period of time.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The compounds of the present invention may also be present in the form of pharmaceutically acceptable salts. For use in medicine, the salts of the compounds of this invention refer to non-toxic “pharmaceutically acceptable salts” (Ref. International J. Pharm. 1986. 33, 201-217; J. Pharm. Sci., 1997 (January), 66, 1, 1). Other salts well known to those in the art may, however, be useful in the preparation of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, ascorbic, tannic, alginic, polyglutamic, naphthalene disulfonic, polygalacturonic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc, salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine.

The present invention includes within its scope prodrugs of the compounds utilized in this invention, e.g., prodrugs of the compounds of Formula (I) and (II). In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. The present invention also includes within its scope metabolites of the compounds utilized in this invention, e.g., metabolites of the compounds of Formula (I) and (II).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

### DETAILED DESCRIPTION

In certain embodiments, the present invention is directed to methods of treating pain (e.g., chronic pain) comprising administering to a patient in need thereof a therapeutically effective amount of a hetero-substituted acetanilide compound.

In certain embodiments, the hetero-substituted acetanilide compound is an adenosine A3 receptor modulator or has a presumptive mechanism of action of an adenosine A3 receptor modulator. As used herein “modulator” is any ligand that binds to the receptors and thus alters (e.g., by inhibiting or activating) the proportion of the receptors, which are in active form, resulting in a biological response.

The term “adenosine A3 receptor” or “A3 AR” refers to a native adenosine A3 receptor or a variant thereof. A3 AR variants include proteins substantially homologous to a native A3AR, i.e., proteins having one or more naturally or non-naturally occurring amino acid deletions, insertions or substitutions (e.g., A3AR derivatives, homologs and fragments), as compared to the amino acid sequence of a native A3 AR. The amino acid sequence of a A3AR variant is at least about 80% identical, at least about 90% identical, or at least about 95% identical to a native A3AR.

The terms “an adenosine A3-mediated condition, disorder or disease” and “a condition, disorder, or disease mediated by A3 AR” refer to a condition, disorder, or disease characterized by abnormal or dysregulated, e.g., greater than normal, A3 AR activity.

A therapeutically effective amount refers to the amount of the hetero-substituted acetanilide compound that is required to confer a therapeutic effect on the chronic pain of the treated subject (e.g., to prevent, alleviate or ameliorate chronic pain) or to confer a therapeutic effect on the neuropathic pain (whether acute or chronic) of the treated subject. Therapeutically effective doses will vary, as recognized by those skilled in the art, depending on the desired outcome, route of administration, excipient usage, and the possibility of co-usage with other therapeutic treatment (e.g., additional active agents). Dosage amount and interval may be adjusted individually to provide plasma levels of the active moiety which are sufficient to maintain the therapeutic effect and/or to avert the adverse side effects, termed the minimal effective concentration (MEC), which depend on individual characteristics and route of administration. HPLC assays or bioassays can be used to determine plasma concentrations. Dosage intervals can also be determined using the MEC value. Preparations should be administered using a regimen, which maintains plasma levels, e.g., above the MEC for 10-90% of the time, between 30-90% or between 50-90%. The exact formulation, route of administration and dosage can be chosen by the individual physician in view of the patient's condition.

Determination of a therapeutically effective amount is within the capability of those skilled in the art, especially in light of the detailed disclosure provided herein. Further, for any compounds used in the methods of the invention, the therapeutically effective amount or dose can be estimated initially from activity assays in cell cultures and/or animals. For example, a dose can be formulated in animal models to achieve a circulating concentration range that includes the IC50 as determined by activity assays. Such information can be used to more accurately determine useful doses in humans. Additionally, toxicity and therapeutic efficacy of the compounds can be determined by standard pharmaceutical procedures in experimental animals, e.g., by determining the IC50 and the LD50 (lethal dose causing death in 50% of the tested animals) for a subject compound. The data obtained from these activity assays and animal studies can be used in formulating a range of dosage for use in human.

In certain embodiments, one or more of the hetero-substituted acetanilide compounds may be administered prophylactically before a patient experiences pain (e.g., chronic pain, acute neuropathic pain). For instance, one or more of the hetero-substituted acetanilide compound may be administered about 48 hours, about 36 hours, about 24 hours, about 12 hours, about 10 hours, about 8 hours, about 6 hours, about 4 hours, about 3 hours, about 2 hours, about 1 hour, or about 0.5 hours before the anticipated onset of pain.

In other embodiments, one or more of the hetero-substituted acetanilide compounds may be administered in response to pain (e.g., chronic pain, acute neuropathic pain). For instance, one or more of the hetero-substituted acetanilide compounds may be administered about 0.5 hour, about 1 hour, about 2 hours, about 3 hours, about 4 hours, about 6 hours, about 8 hours, about 10 hours, about 12 hours, about 24 hours, about 36 hours, or about 48 hours after the onset of pain (e.g., chronic pain, acute neuropathic pain).

The term “chronic pain,” as used herein refers to pain which persists over an extended duration, e.g., over three months or longer. Any reference to chronic pain throughout this disclosure encompasses, among others, neuropathic pain, musculoskeletal pain, nociplastic pain, nociceptive pain, and non-nociceptive pain.

The term “neuropathic pain,” as used herein refers to pain caused by damage, dysfunction, or disease affecting somatosensory nervous system which may result from nerve damage and/or due to disorders of the peripheral nervous system and/or the central nervous system (brain and spinal cord). Neuropathic pain may be divided into peripheral neuropathic pain, central neuropathic pain, or mixed (peripheral and central) neuropathic pain. Neuropathic pain may be the result of a number of disease processes and may be due to damage in a number of locations. Exemplary, non-limiting, causes of central neuropathic pain include spinal cord injury, multiple sclerosis, low back pain with sciatica, and some strokes. Exemplary, non-limiting, causes of peripheral neuropathic pain include back pain (due to nerve injury or dysfunction), phantom pain, complex regional pain syndrome type II (CRPS II), trigeminal neuralgia, diabetes (e.g., Diabetic peripheral neuropathy (DPN)), metabolic conditions, postherpetic neuralgia (PHN), herpes zoster infection, HIV-related neuropathies, nutritional deficiencies, toxins, remote manifestations of malignancies, immune mediated disorders, bladder pain, and physical trauma to nerve trunk. Neuropathic pain is common in cancer as a direct result of cancer on peripheral nerves (e.g., compression by a tumor), or as a side effect of chemotherapy, radiation injury, or surgery. Neuropathic pain, as used herein, may refer to both, acute neuropathic pain and chronic neuropathic pain in duration. Neuropathic symptoms include, without limitations, paresthesia and/or dysesthesia (e.g., abnormal nonpainful sensations such as tingling, numbness, pins, and needles), allodynia, hyperalgesia, neurological sensory deficits in the painful area together with other deficits (motor, cognitive, and so on).

The term “nociceptive pain,” as used herein refers to pain arising from physical damage or potential damage to the body (e.g., to the nociceptive nerve fibers) and may be triggered by inflammation, chemicals, or physical events. Exemplary, non-limiting, causes of nociceptive pain include sports injury, inflammatory and/or joint pain (e.g., arthritis), tissue damage. Examples of nociceptive pain include, without limitations, osteoarthritis, visceral pain, headache, ischemic pain, cancer pain (without nerve injury), back pain (without nerve injury).

The term “nociplastic pain,” as used herein refers to pain that arises from altered nociception despite no clear evidence of actual or threatened tissue damage causing the activation of peripheral nociceptors or evidence for disease or lesion of the somatosensory system causing the pain. Patients can have a combination of nociceptive and nociplastic pain.

The term “non-nociceptive and non-neuropathic pain,” as used herein refers to pain arising without any known tissue or nerve damage and is not considered neuropathic pain or nociceptive pain.

In certain embodiments, the pain treated with the compositions disclosed herein can be due to mixed nociceptive and neuropathic pain syndromes. Examples of mixed nociceptive and neuropathic pain syndromes include, without limitations, chronic back pain (e.g., nerve lesion or dysfunction combined with nociceptive activation from ligaments, joints, muscles, tendons), cancer pain (with nerve infiltration), complex regional pain syndrome type I (CRPS I) without nerve injury.

In certain embodiments, the pain treated with the composition disclosed herein can be due to, e.g., fibromyalgia, musculoskeletal disorders such as osteoarthritis/degenerative joint disease/spondylosis, rheumatoid arthritis, lyme disease, reiter syndrome, disk hemiation/facet osteoarthropathy, fractures/compression fracture of lumbar vertebrae, faulty or poor posture, fibromyalgia, polymyalgia rheumatica, mechanical low back pain, chronic coccygeal pain, muscular strains and sprains, pelvic floor myalgia (levator ani spasm), piriformis syndrome, rectus tendon strain, hernias (e.g., obturator, sciatic, inguinal, femoral, spigelian, perineal, umbilical), abdominal wall myofascial pain (trigger points), chronic overuse syndromes (e.g., tendinitis, bursitis), neurological disorders such as, brachial plexus traction injury, cervical radiculopathy, thoracic outlet syndrome, spinal stenosis, arachnoiditis syndrome, metabolic deficiency myalgias, polymyositis, neoplasia of spinal cord or sacral nerve, cutaneous nerve entrapment in surgical scar, postherpetic neuralgia (shingles), neuralgia (e.g., iliohypogastric, ilioinguinal, or genitofemoral nerves), polyneuropathies, polyradiculoneuropathies, mononeuritis multiplex, chronic daily headaches, muscle tension headaches, migraine headaches, temporomandibular joint dysfunction, temporalis tendonitis, sinusitis, atypical facial pain, trigeminal neuralgia, glossopharyngeal neuralgia, nervus intermedius neuralgia, sphenopalatine neuralgia, referred dental or temporomandibular joint pain, abdominal epilepsy, abdominal migraine, urologic disorders, bladder neoplasm, chronic urinary tract infection, interstitial cystitis, radiation cystitis, recurrent cystitis, recurrent urethritis, urolithiasis, uninhibited bladder contractions (detrusor-sphincter dyssynergia), bladder pain syndrome, urethral diverticulum, chronic urethral syndrome, urethral carbuncle, prostatitis, urethral stricture, testicular torsion, peyronie disease, gastrointestinal disorders such as chronic visceral pain syndrome, gastroesophageal reflux, peptic ulcer disease, pancreatitis, chronic intermittent bowel obstruction, colitis, chronic constipation, diverticular disease, inflammatory bowel disease, irritable bowel syndrome; reproductive disorders (extrauterine) such as endometriosis, adhesions, adnexal cysts, chronic ectopic pregnancy, chlamydial endometritis or salpingitis, endosalpingiosis, ovarian retention syndrome (residual ovary syndrome), ovarian remnant syndrome, ovarian dystrophy or ovulatory pain, pelvic congestion syndrome, postoperative peritoneal cysts, residual accessory ovary, subacute salpingo-oophoritis, tuberculous salpingitis; reproductive disorders (uterine) such as adenomyosis, chronic endometritis, atypical dysmenorrhea or ovulatory pain, cervical stenosis, endometrial or cervical polyps, leiomyomata, symptomatic pelvic relaxation (genital prolapse), intrauterine contraceptive device, labor pain; psychological disorders such as bipolar personality disorders, depression, porphyria, sleep disturbances; and other conditions such as cardiovascular disease (e.g., angina), myocardial infarction pain, pancreatic pain, colic pain, post-operative pain, muscle pain, arthiritic pain, pain associated with a periodontal disease (e.g., ginigivitis and periodontitis), intraocular pain, peripheral vascular disease and chemotherapeutic, radiation, or surgical complications.

In certain embodiments, one or more of the hetero-substituted acetanilide compounds may be administered prophylactically before a patient experiences inflammation (e.g., local inflammation or systemic inflammation). For instance, one or more of the hetero-substituted acetanilide compounds may be administered about 48 hours, about 36 hours, about 24 hours, about 12 hours, about 10 hours, about 8 hours, about 6 hours, about 4 hours, about 3 hours, about 2 hours, about 1 hour, or about 0.5 hours before the anticipated onset of inflammation.

In other embodiments, one or more of the hetero-substituted acetanilide compounds may be administered in response to inflammation (e.g., local inflammation or systemic inflammation). For instance, one or more of the hetero-substituted acetanilide compounds may be administered about 0.5 hour, about 1 hour, about 2 hours, about 3 hours, about 4 hours, about 6 hours, about 8 hours, about 10 hours, about 12 hours, about 24 hours, about 36 hours, or about 48 hours after the onset of inflammation (e.g., local inflammation or systemic inflammation).

For example, one or more of the hetero-substituted acetanilide compounds described herein can be used to treat or prevent inflammatory diseases including, but not limited to, organ transplant rejection; reoxygenation injury resulting from organ transplantation including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory bowel diseases, such as ileitis, ulcerative colitis, Barrett's syndrome, and Crohn's disease; inflammatory lung diseases, such as asthma, adult respiratory distress syndrome, and chronic obstructive airway disease; inflammatory diseases of the eye, including corneal dystrophy, trachoma, onchocerciasis, uveitis, sympathetic ophthalmitis and endophthalmitis; chronic inflammatory disease of the gum, including gingivitis and periodontitis; tuberculosis; leprosy; inflammatory diseases of the kidney, including uremic complications, glomerulonephritis and nephrosis; inflammatory disease of the skin, including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, including Type I and Type II diabetes mellitus; diabetic complications, including, but not limited to, diabetic cataract, glaucoma, retinopathy, nephropathy (such as microalbuminuria and progressive diabetic nephropathy), gangrene of the feet, atherosclerotic coronary arterial disease, peripheral arterial disease, nonketotic hyperglycemic-hyperosmolar coma, foot ulcers, joint problems, and a skin or mucous membrane complication (such as an infection, a shin spot, a candidal infection or necrobiosis lipoidica diabeticorum), immune-complex vasculitis, and systemic lupus erythematosus (SLE); inflammatory disease of the heart, such as cardiomyopathy, ischemic heart disease hypercholesterolemia, and artherosclerosis; as well as various other diseases that can have significant inflammatory components, including preeclampsia, chronic liver failure, brain and spinal cord trauma, and cancer.

In certain embodiments, one or more of the hetero-substituted acetanilide compounds described herein can also be used to treat or prevent pain associated with inflammatory disease that can, for example, be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

In certain embodiments, one or more of the hetero-substituted acetanilide compounds described herein can be used in a method of treating or preventing a disease, state, or condition by, e.g., modulating an A3 adenosine receptor (e.g., activating or inhibiting the A3 adenosine receptor), wherein the disease, state, or condition may be selected from the group consisting of neuropathic pain, vascular inflammation, arthritis, allergies, asthma, wound healing, stroke, cardiac failure, acute spinal cord injury, acute head injury or trauma, seizure, neonatal hypoxia, cerebral palsy, chronic hypoxia due to arteriovenous malformations and occlusive cerebral artery disease, ischemia and reperfusion injury in skeletal muscle, severe neurological disorders related to excitotoxicity, Parkinson's disease, Huntington's chorea, diseases of the CNS, cardiac disease, kidney disease, glaucoma, cancer, neuropathic pain, neuropathic pain associated with diabetes, transient ischemic attacks, myeloprotection, dry eye syndrome, osteoarthritis, rheumatoid arthritis, loss of skin pigmentation, inflammatory bowel disease, pulmonary inflammation, uveitis, and septic shock.

In certain embodiments, the disclosure provides for a method of treating or preventing neuropathic pain in a patient in need thereof. In another embodiment, the disclosure provides a method of treating or preventing postoperative pain in a patient in need thereof. In yet another embodiment, the disclosure provides a method of treating or preventing chronic or persistent pain in a patient in need thereof. In certain embodiments, the disclosure provides a method of treating or preventing pain associated with chemotherapy-induced peripheral neuropathy (CIPN) in a patient in need thereof.

In certain embodiments, the disclosure provides for a method of treating or preventing or alleviating the symptoms of neuropathic pain regardless of the cause of the pain, for example, but not limited to, spinal cord injury, multiple sclerosis, stroke, diabetes, herpes zoster infection, HIV-related neuropathies, nutritional deficiencies, toxins, remote manifestations of malignancies, genetic, immune mediated disorders or physical trauma to a nerve trunk, cancer, chemotherapy, radiation injury or surgery (e.g., postoperative pain), vulvodynia, and burning mouth syndrome. In an embodiment, the neuropathic pain may be associated with chronic use of opioids.

In certain embodiments, one or more of the hetero-substituted acetanilide compounds described herein may be administered in relation to a particular condition that is being treated without a significant potential for addiction, abuse, misuse, drowsiness, and/or respiratory depression, e.g., compared to opioids.

In certain embodiments, one or more of the hetero-substituted acetanilide compounds described herein may be administered in relation to a particular condition that is being treated without a significant potential for cardiovascular adverse events (e.g., thrombotic events), serious gastrointestinal adverse events, and/or renal adverse events, e.g., compared to NSAIDs.

In certain embodiments, one or more of the hetero-substituted acetanilide compounds described herein may be administered in relation to a particular condition that is being treated without a significant potential for hepatic adverse events (e.g., acute liver failure), e.g., compared to acetaminophen.

Currently known analgesics include, but are not limited to, opioids, morphinomimetics, antidepressants, antiepileptics, NMDA receptor antagonists, fatty acid amine hydrolyase inhibitors, anticonvulsives, non-steroidal anti-inflammatory drugs (NSAIDs), COX-2 inhibitors, NOS inhibitors, acetaminophen, and calcium channel subunit α₂δ ligands.

Example opioids include any natural or synthetic opioid analgesic, such as morphine, fentanyl, codeine, thebaine, diacetylmorphine (heroin), dihydrocodeine, hydrocodone, hydromorphone, nicomorphine, oxycodone, oxymorphone, alphamethylfentanyl, alfentanil, sufentanil, remifentanil, carfentanyl, ohmefentanyl, nocaine, pethidine (meperidine), ketobemidone, MPPP, allylprodine, prodine, PEPAP, propoxyphene, dextropropoxyphene, dextromoramide, bezitramide, piritramide, methadone, dipipanone, levoalphacetylmethadol (LAAM), loperamide, diphenoxylate, pentazocine, phenazocine, buprenorphine, etorphine, butorphanol, nalbuphine, levorphanol, levomethorphan, dezocine, lefetamine, tilidine, tramadol, propoxyphene, and oxycodone. As intended herein, an opioid also encompasses any natural or synthetic narcotic antagonist such as nalmefene, naloxone or naltrexone as well as any natural or synthetic mixed opioid agonist/antagonist such as nalbuphine, butorphanol, buprenorphine and pentazocine.

Example non-steroidal anti-inflammatory drugs (NSAIDs) include aspirine, ibuprofen, acetaminophen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lomoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, and licofelone. Example antidepressants include tricyclic antidepressants such as: amitriptyline, amitriptylinoxide, butriptyline, clomipramine, demexiptiline, desipraminc, dibenzepin, dimetacrine, dosulepin, doxepin, imipramine, imipraminoxide, lofepramine, melitracen, metapramine, nitroxazepine, nortriptyline, noxiptiline, pipofezine, propizepine, protriptyline, and quinupramine; amineptine, norepinephrine, iprindole, opipramol, tianeptine, trimipramine, carbamezapine, and flupirtine.

In certain embodiments, the one or more of the hetero-substituted acetanilide compounds described herein may be administered in methods of treating and/or preventing and/or ameliorating pain (e.g., chronic pain, neuropathic pain, etc.) and/or inflammation (e.g., local inflammation, systemic inflammation, etc.), which do not respond to acetaminophen. In certain embodiments, the one or more of the hetero-substituted acetanilide compounds described herein may be administered in methods of treating and/or preventing and/or ameliorating a particular condition, disease state, or symptoms thereof which would not respond to acetaminophen.

In certain embodiments, the use of one or more of the hetero-substituted acetanilide compounds described herein in a method of treating and/or preventing and/or ameliorating a particular condition and/or disease state and/or symptoms thereof, exhibits enhanced safety as compared to the administration of a different analgesic currently used for said condition.

In certain embodiments, the use of one or more of the hetero-substituted acetanilide compounds described herein in a method of treating and/or preventing and/or ameliorating pain (e.g., chronic pain, neuropathic pain, etc) exhibits lower hepatotoxicity as compared to the administration of acetaminophen.

In certain embodiments, the use of one or more of the hetero-substituted acetanilide compounds described herein in a method of treating and/or preventing and/or ameliorating pain (e.g., chronic pain, neuropathic pain, etc) exhibits enhanced safety in one or more of: gastrointestinal safety, cardiovascular safety, respiratory safety, central nervous system safety, and/or renal safety, as compared to the administration of other classes of analgesics or co-analgesics (such as opioids, NSAIDS, cannabinoids, gabapentinoids, etc).

In certain embodiments, the instant disclosure is directed to a method of treating and/or preventing and/or ameliorating pain (e.g., chronic pain, neuropathic pain, etc) with the benefit of decreased incidences of hepatic adverse events (e.g., reduced incidences of one or more of elevations in aspartate aminotransferase (AST) levels and/or (alanine transaminase) ALT levels and/or blood bilirubin levels), the method comprising administering an effective amount of one or more of the hetero-substituted acetanilide compounds described herein to a patient in need thereof. In certain embodiments, the reduced incidences of hepatic adverse events may be compared to the hepatic adverse events experienced by patients receiving a comparative dose of, e.g., acetaminophen, where a comparative dose may refer to a comparative dose to attain the same analgesic effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of gastrointestinal adverse events (e.g., reduced incidences of one or more of hypoaesthesia oral, nausea, diarrhea, vomiting, and/or abdominal pain), the method comprising administering an effective amount of one or more of the hetero-substituted acetanilide compounds described herein to a patient in need thereof. In certain embodiments, the reduced incidences of gastrointestinal adverse events may be compared to the gastrointestinal adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of nervous system disorders adverse events (e.g., reduced incidences of one or more of headache, dizziness, paraesthesia, syncope), the method comprising administering an effective amount of one or more of the hetero-substituted acetanilide compounds described herein to a patient in need thereof. In certain embodiments, the reduced incidences of nervous system disorders adverse events may be compared to the nervous system disorders adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of general disorders adverse events (e.g., reduced incidences of one or more of pyrexia, chills, feeling hot, malaise, suprapubic pain), the method comprising administering an effective amount of one or more of the hetero-substituted acetanilide compounds described herein to a patient in need thereof. In certain embodiments, the reduced incidences of general disorders adverse events may be compared to the general disorders adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of respiratory, thoracic and mediastinal disorders adverse events (e.g., reduced incidences of oropharyngeal pain), the method comprising administering an effective amount of one or more of the hetero-substituted acetanilide compounds described herein to a patient in need thereof. In certain embodiments, the reduced incidences of respiratory, thoracic and mediastinal disorders adverse events may be compared to the respiratory, thoracic and mediastinal disorders adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of cardiovascular adverse events, the method comprising administering an effective amount of one or more of the hetero-substituted acetanilide compounds described herein to a patient in need thereof. In certain embodiments, the reduced incidences of cardiovascular adverse events may be compared to the cardiovascular adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of respiratory adverse events, the method comprising administering an effective amount of one or more of the hetero-substituted acetanilide compounds described herein to a patient in need thereof. In certain embodiments, the reduced incidences of respiratory adverse events may be compared to the respiratory adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of central nervous system (CNS) adverse events, the method comprising administering an effective amount of one or more of the hetero-substituted acetanilide compounds described herein to a patient in need thereof. In certain embodiments, the reduced incidences of CNS adverse events may be compared to the CNS adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) and/or inflammation and/or an inflammatory condition with the benefit of decreased incidences of renal adverse events, the method comprising administering an effective amount of one or more of the hetero-substituted acetanilide compounds described herein to a patient in need thereof. In certain embodiments, the reduced incidences of renal adverse events may be compared to the renal adverse events experienced by patients receiving a comparative dose of, e.g., an opioid and/or an NSAID, where a comparative dose may refer to a comparative dose to attain the same analgesic and/or anti-inflammatory effect.

In certain embodiments, the instant disclosure is directed to a method of treating and/or preventing and/or ameliorating pain (e.g., chronic pain, neuropathic pain, etc) while reducing the amount of opioids (i.e., opioid sparing) administered to the patient, the method comprising administering an effective amount of one or more of the hetero-substituted acetanilide compounds described herein to a patient in need thereof.

In certain embodiments, the instant disclosure is directed to a method of treating and/or preventing and/or ameliorating pain (e.g., chronic pain, neuropathic pain, etc) while reducing the amount of NSAIDs (i.e., NSAID sparing) administered to the patient, the method comprising administering an effective amount of one or more of the hetero-substituted acetanilide compounds described herein to a patient in need thereof.

In certain embodiments, the one or more of the hetero-substituted acetanilide compounds described herein may be used in a method of treating and/or preventing and/ameliorating an adenosine A3-mediated condition, disorder or disease.

In certain embodiments, the adenosine A3-mediated conditions, disorders, or diseases include, but are not limited to, myeloproliferative disorders such as polycythemia vera (PCV), essential thrombocythemia and idiopathic myelofibrosis (IMF); leukemia such as myeloid leukemia including chronic myeloid leukemia (CML), imatinib-resistant forms of CML, acute myeloid leukemia (AML), and a subtype of AML, acute megakaryoblastic leukemia (AMKL); lymphoproliferative diseases such as myeloma; cancer including head and neck cancer, prostate cancer, breast cancer, ovarian cancer, melanoma, lung cancer, brain tumor, pancreatic cancer and renal carcinoma; and inflammatory diseases or disorders related to immune dysfunction, immunodeficiency, immunomodulation, autoimmune diseases, tissue transplant rejection, graft-versus-host disease, wound healing, kidney disease including diabetic neuropathy, multiple sclerosis, thyroiditis, type I diabetes, sarcoidosis, psoriasis, allergic rhinitis, inflammatory bowel disease including Crohn's disease and ulcerative colitis (UC), systemic lupus erythematosis (SLE), arthritis, osteoarthritis, rheumatoid arthritis, osteoporosis, asthma and chronic obstructive pulmonary disease (COPD) and dry eye syndrome (or keratoconjunctivitis sicca (KCS)).

In certain embodiments, provided herein are methods of using the one or more of the hetero-substituted acetanilide compounds described herein, for the treatment, prevention, or amelioration of a disease or disorder selected from myeloproliferative disorders such as polycythemia vera (PCV), essential thrombocythemia and idiopathic myelofibrosis (IMF) and hypereosinophilic syndrome (HES); leukemia such as myeloid leukemia including chronic myeloid leukemia (CML), imatinib-resistant forms of CML, acute myeloid leukemia (AML), acute lymphoblastic leukemia (ALL) and a subtype of AML, acute megakaryoblastic leukemia (AMKL); lymphoproliferative diseases such as myeloma; cancer including head and neck cancer, prostate cancer, breast cancer, ovarian cancer, melanoma, lung cancer, brain cancer, pancreatic cancer, gastric cancer, thyroid cancer, renal carcinoma, Kaposi's sarcoma, Castleman's disease, melanoma.

In certain embodiments, provided herein are methods of using the one or more of the hetero-substituted acetanilide compounds described herein, for the treatment, prevention, or amelioration of a disease or disorder selected from inflammatory diseases or disorders related to immune dysfunction, immunodeficiency or immunomodulation, such as tissue transplant rejection, graft-versus-host disease, wound healing, kidney disease; autoimmune diseases such as multiple sclerosis, thyroiditis, type I diabetes, sarcoidosis, psoriasis, allergic rhinitis, atopic dermatitis, myasthenia gravis, inflammatory bowel disease including Crohn's disease and ulcerative colitis (UC), systemic lupus erythematosis (SLE), arthritis, osteoarthritis, rheumatoid arthritis, osteoporosis, asthma and chronic obstructive pulmonary disease (COPD), inflammatory diseases of the eye including conjunctivitis, uveitis, iritis, scleritis, inflammatory diseases of the respiratory tract including the upper respiratory tract such as rhinitis and sinusitis and inflammatory diseases of the lower respiratory tract including bronchitis; inflammatory myopathy such as myocarditis, other inflammatory diseases such as ischemia reperfusion injuries related to an inflammatory ischemic event such as a stroke or cardiac arrest, and other inflammatory conditions such as systemic inflammatory response syndrome (SIRS) and sepsis.

In certain embodiments, adenosine A3-mediated diseases and disorders include restenosis, fibrosis and scleroderma. In certain embodiments, adenosine A3-mediated diseases include viral diseases such as Epstein Barr virus (EBV), hepatitis (hepatitis B or hepatitis C), human immunodeficiency virus (HIV), Human T-lymphotropic virus type 1 (HTLV-1), varicella-zoster virus and the human papilloma virus (HPV).

In certain embodiments, the adenosine A3-mediated condition, disorder, or disease is a cardiovascular disease, including, but not limited to, ischaemic heart disease. In certain embodiments, the adenosine A3-mediated condition, disorder, or disease is atherosclerosis. In certain embodiments, the adenosine A3-mediated condition, disorder, or disease is lung injury. In certain embodiments, the adenosine A₃-mediated condition, disorder, or disease is renal failure. In certain embodiments, the adenosine A3-mediated condition, disorder, or disease is an eye disease, including, but not limited to, glaucoma and ocular hypertension. In certain embodiments, the adenosine A3-mediated condition, disorder, or disease is colon cancer or multidrug resistant cancer.

In certain embodiments, the administration route of the hetero-substituted acetanilide compound includes routes such as oral, intravenous, nasal, inhalational, topical, buccal, rectal, pleural, peritoneal, vaginal, intramuscular, subcutaneous, transdermal, epidural, intratrachael, otic, intraocular, intraarticular instillation, or intrathecal. In one embodiment, the administration route of the hetero-substituted acetanilide compound is topical via, e.g., creams, gels, or transdermal delivery systems (e.g., skin patches). In one embodiment, the administration route of the hetero-substituted acetanilide compound is oral. In one embodiment, the administration route of the hetero-substituted acetanilide compound is parenteral. In one embodiment, the administration route of the hetero-substituted acetanilide compound is intranasal vita suitable intranasal vehicles.

Pharmaceutical compositions used in the present invention may be administered in a single daily dose, or the total daily dosage may be administered in divided doses of two, three or four times daily. In one embodiment, the pharmaceutical compositions used in the present invention maintain their efficacy over an extended duration and may be administered once a day in a single daily dose. In certain embodiments, the pharmaceutical compositions used in the present invention maintain their efficacy over and extended duration (e.g., over 24 hours) and may be administered once every 36 hours, once every 48 hours, once every 60 hours, once every 72 hours, once every 84 hours, once every 96 hours, once every 108 hours, once every 120 hours, once every 132 hours, once every 144 hours, once every 156 hours, or once every 168 hours.

In certain embodiments, a single dose of the pharmaceutical compositions used in the present disclosure provide effective anti-inflammatory response for a duration of at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, or at least about 24 hours. In certain embodiments, a single dose of the pharmaceutical compositions used in the present disclosure provide effective anti-inflammatory response for a duration of up to about 72 hours, up to about 48 hours, up to about 24 hours, up to about 20 hours, up to about 16 hours, up to about 12 hours, up to about 8 hours, up to about 4 hours, or up to about 2 hours.

In certain embodiments, a single dose of the pharmaceutical compositions used in the present disclosure provide effective analgesia for a duration of at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, or at least about 24 hours. In certain embodiments, a single dose of the pharmaceutical compositions used in the present disclosure provide effective analgesia for a duration of up to about 72 hours, up to about 48 hours, up to about 24 hours, up to about 20 hours, up to about 16 hours, up to about 12 hours, up to about 8 hours, up to about 4 hours, or up to about 2 hours. In certain embodiments, effective analgesia may be inferred from pain intensity differences (PID) assessment over a particular time duration after administration of the pharmaceutical composition.

Without being construed as limiting, it is believed that the effective analgesia and/or effective anti-inflammatory response is maintained for an extended duration, in certain embodiments, due to either pharmacokinetic properties (long half-life of the one or more of the hetero-substituted acetanilide compounds described herein), or due to the mechanism of action of the one or more of the hetero-substituted acetanilide compounds described herein (e.g., modulation of a receptor such as the adenosine A3 receptor, resulting in a biological response after which the subject no longer experiences pain and/or inflammation).

The hetero-substituted acetanilide compound can be used in the methods of the present invention in an amount, e.g., from any of about 0.01 mg, about 1 mg, about 5 mg, about 10 mg, about 25 mg, about 50 mg, about 75 mg, about 100 mg, about 150 mg, about 200 mg, about 250 mg, about 300 mg, about 350 mg, about 400 mg, about 450 mg, or about 500 mg to any of about 600 mg, about 700 mg, about 800 mg, about 900 mg, about 1000 mg, about 1200 mg, about 1400 mg, about 1500 mg, about 1750 mg, about 2000 mg, about 2500 mg, about 3000 mg, about 3500 mg, about 4000 mg, about 4500 mg, or about 5000 mg of active ingredient per day in a single or divided dose (e.g., twice, three or four times daily). In certain embodiments, the hetero-substituted acetanilide compound can be used in the methods of the present invention in an amount about 0.01 mg to about 15,000 mg, from about 1 mg to about 5000 mg, from about 500 mg to about 4000 mg or from about 1 mg to about 500 mg of active ingredient per day in a single or divided dose (e.g., twice, three or four times daily). For oral administration, the amount of active ingredient in a single dose (which may be administered once, twice, thrice, or four times daily) can be, e.g., from about 0.01 mg to about 2000 mg, or about 1 mg, about 5 mg, about 10 mg, about 50 mg, about 100 mg, about 150 mg, about 200 mg, about 250 mg, about 500 mg, about 1000 mg, about 1500 mg, or about 2000 mg. In certain embodiments, the doses described herein may be for an average (70 kg) human. In certain embodiments, the dose may be modified by weight of the patient based on an average (70 kg) human as the baseline.

In one embodiment, the hetero-substituted acetanilide compound can be orally administered in the methods of the present invention in an amount of about 300 mg once a day. In one embodiment, the hetero-substituted acetanilide compound can be orally administered in the methods of the present invention in an amount of about 500 mg once a day. In one embodiment, the hetero-substituted acetanilide compound can be orally administered in the methods of the present invention in an amount of about 750 mg once a day.

In one embodiment, the hetero-substituted acetanilide compound can be orally administered in the methods of the present invention in an amount of about 300 mg twice a day. In one embodiment, the hetero-substituted acetanilide compound can be orally administered in the methods of the present invention in an amount of about 500 mg twice a day. In one embodiment, the hetero-substituted acetanilide compound can be orally administered in the methods of the present invention in an amount of about 750 mg twice a day.

In one embodiment, the hetero-substituted acetanilide compound can be orally administered in the methods of the present invention in an amount of about 1000 mg once a day. In one embodiment, the hetero-substituted acetanilide compound can be orally administered in the methods of the present invention in an amount of about 2000 mg once a day. In one embodiment, the hetero-substituted acetanilide compound can be orally administered in the methods of the present invention in an amount of about 1000 mg twice a day. In one embodiment, the hetero-substituted acetanilide compound can be orally administered in the methods of the present invention in an amount of about 2000 mg twice a day.

In certain embodiments, the hetero-substituted acetanilide compound for use in treatment of chronic pain (e.g., chronic neuropathic pain), acute neuropathic pain, inflammation (e.g., local inflammation or systemic inflammation), an inflammatory response, or an inflammatory condition is selected from Formula (I), Formula (11), or a combination thereof, where Formula (I) and Formula (II) are described in more detail below.

In certain embodiments, the compound of Formula (I) is:

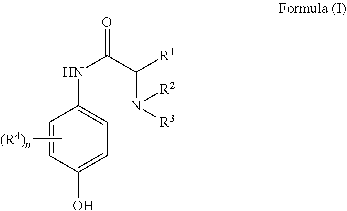

wherein:

R¹ and R² taken together with the atoms to which they are attached form a radical selected from the group consisting of a 5 to 10 membered cycloheteroalkanyl, 5 to 10 membered cycloheteroalkenyl, a 5 to 10 membered heteroaryl, and a 5 to 10 membered cycloheteroalkyl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R³ is hydrogen or C₁₋₆alkanyl;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆alkanylamino and cyano;

n is an integer from 1 to 3; and

enantiomers, diastereomers, tautomers, solvates, or pharmaceutically acceptable salts thereof.

Embodiments of the present invention include uses of compositions comprising compounds of formula (I) for treatment of chronic pain (e.g., chronic neuropathic pain), acute neuropathic pain, inflammation (e.g., local inflammation or systemic inflammation), an inflammatory response, or an inflammatory condition, wherein for formula (I):

a) R¹ and R² taken together with the atoms to which they are attached form a 5 to 10 membered cycloheteroalkanyl radical, wherein, in addition to the heteroatom N, said radical may optionally contain 1 to 3 additional heteroatoms, independently selected from the group consisting of O. N and S; additionally, said radical may be further optionally substituted with one to three substituents independently selected from the group consisting of C₁₋₆alkanyl and oxo;

b) R¹ and R^{z} taken together with the atoms to which they are attached form a 5 to 10 membered cycloheteroalkenyl radical, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

c) R¹ and R² taken together with the atoms to which they are attached form a 5 to 10 membered heteroaryl radical, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

d) R¹ and R² taken together with the atoms to which they are attached form a radical selected from the group consisting of a 5 to 10 membered cyclic heteroalkyl and a 5 to 10 membered heteroaryl; additionally, said radical may be further optionally substituted with one to three substituents independently selected from the group consisting of C₁₋₆alkanyl and oxo;

e) R³ is hydrogen;

f) R⁴ is hydrogen:

g) n is 1; and

h) combinations of a) through g) above.

An embodiment of the present invention is directed to use of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a 5 to 10 membered cycloheteroalkanyl radical, wherein, in addition to the heteroatom N, said radical may optionally contain 1 to 3 additional heteroatoms, independently selected from the group consisting of O. N and S; additionally, said radical may be further optionally substituted with one to three substituents independently selected from the group consisting of C₁₋₆alkanyl and oxo;

R³ is hydrogen or C₁₋₆alkanyl;

R⁴ is a substituent selected from the group consisting of hydrogen, C₁₋₆ alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano,

n is an integer from 1 to 3; and

enantiomers, diastereomers, tautomers, solvates, or pharmaceutically acceptable salts thereof.

Another embodiment of the present invention is directed to use of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, w % herein:

R¹ and R² taken together with the atoms to which they are attached form a 5 to 10 membered cycloheteroalkenyl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R³ is hydrogen or C₁₋₆alkanyl;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆alkanylamino and cyano;

n is an integer from 1 to 3; and

enantiomers, diastereomers, tautomers, solvates, or pharmaceutically acceptable salts thereof.

Still another embodiment of the present invention is directed to uses of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a 5 to 10 membered heteroaryl radical, wherein, in addition to the heteroatom N, said radical may optionally contain 1 to 3 additional heteroatoms, independently selected from the group consisting of O. N and S; additionally, said radical may be further optionally substituted with one to three substituents independently selected from the group consisting of C₁₋₆alkanyl and oxo;

R³ is hydrogen or C₁₋₆alkanyl;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is an integer from 1 to 3; and

enantiomers, diastereomers, tautomers, solvates, or pharmaceutically acceptable salts thereof.

Still another embodiment of the present invention is directed to uses of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl; additionally, said radical may be further optionally substituted with one to three substituents independently selected from the group consisting of C₁₋₆alkanyl and oxo;

R³ is hydrogen or C₁₋₆alkanyl;

R⁴ is a substituent selected from the group consisting of hydrogen, C₁₋₆alkanyl, C₁₋₆ (alkanyloxy, fluorinated alkanyl, fluorinated alkanyloxy, halogen, hydroxyl, nitro, amino, C₁₋₆ alkanylamino; C₁₋₆dialkanylamino and cyano;

n is an integer from 1 to 3; and

enantiomers, diastereomers, tautomers, solvates, or pharmaceutically acceptable salts thereof.

Still yet another embodiment of the present invention is directed to uses of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R³ is hydrogen;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is an integer from 1 to 3; and

enantiomers, diastereomers, tautomers, solvates, or pharmaceutically acceptable salts thereof.

Still another embodiment of the present invention is directed to uses of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O. N and S; additionally, said radical may be further optionally substituted with one to three substituents independently selected from the group consisting of C₁₋₆alkanyl and oxo;

R³ is hydrogen or C₁₋₆alkanyl;

R⁴ is hydrogen;

n is an integer from 1 to 3; and

enantiomers, diastereomers, tautomers, solvates, or pharmaceutically acceptable salts thereof.

Still yet another embodiment of the present invention is directed to uses of compositions comprising a compound of Formula (I) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R³ is hydrogen or C₁₋₆alkanyl;

R⁴ is a substituent selected from the group consisting of hydrogen, C₁₋₆ alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is 1; and

enantiomers, diastereomers, tautomers, solvates, or pharmaceutically acceptable salts thereof.

In certain embodiments, the compound of Formula (II) is:

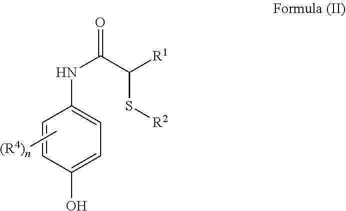

wherein:

R¹ is a substituent independently selected from the group consisting of hydrogen and C₁₋₆alkanyl:

R² is a substituent independently selected from the group consisting of hydrogen, C₁₋₆ alkanyl and dioxo; or

R¹ and R² taken together with the atoms to which they are attached form a radical selected from the group consisting of a 5 to 10 membered cycloheteroalkanyl, 5 to 10 membered cycloheteroalkenyl, a 5 to 10 membered heteroaryl, and a 5 to 10 cycloheteroalkyl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is an integer from 1 to 3; and

enantiomers, diastereomers, tautomers, solvates, or pharmaceutically acceptable salts thereof.

Embodiments of the present invention include uses of compositions comprising compounds of formula (II) for treatment of chronic pain (e.g., chronic neuropathic pain), acute neuropathic pain, inflammation (e.g., local inflammation or systemic inflammation), an inflammatory response, or an inflammatory condition, wherein for formula (II):

a) R¹ and R² taken together with the atoms to which they are attached form a 5 to 10 membered cycloheteroalkanyl, wherein, in addition to the heteroatom N, said radical may optionally contain 1 to 3 additional heteroatoms, independently selected from the group consisting of O. N and S; additionally, said radical may be further optionally substituted with one to three substituents independently selected from the group consisting of C₁₋₆alkanyl and oxo;

b) R¹ and R² taken together with the atoms to which they are attached form a 5 to 10 membered cycloheteroalkenyl radical, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

c) R¹ and R² taken together with the atoms to which they are attached form a 5 to 10 membered heteroaryl radical, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

d) R¹ and R² taken together with the atoms to which they are attached form a radical selected from the group consisting of a 5 to 10 membered cyclic heteroalkyl and a 5 to 10 membered heteroaryl; additionally, said radical may be further optionally substituted with one to three substituents independently selected from the group consisting of C₁₋₆alkanyl and oxo:

e) R⁴ is hydrogen;

f) n is 1; and

g) combinations of a) through f) above.

An embodiment of the present invention is directed to uses of compositions comprising a compound of Formula (II) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a 5 to 10 membered cycloheteroalkanyl radical, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is an integer from 1 to 3; and

enantiomers, diastereomers, tautomers, solvates, or pharmaceutically acceptable salts thereof.

Another embodiment of the present invention is directed to uses of compositions comprising a compound of Formula (II) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a 5 to 10 membered cyclic heteroalkenyl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is an integer from 1 to 3; and

enantiomers, diastereomers, tautomers, solvates, or pharmaceutically acceptable salts thereof.

Still another embodiment of the present invention is directed to uses of compositions comprising a compound of Formula (II) treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a 5 to 10 membered heteroaryl radical, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano:

n is an integer from 1 to 3; and

enantiomers, diastereomers, tautomers, solvates, or pharmaceutically acceptable salts thereof.

Still another embodiment of the present invention is directed to uses of compositions comprising a compound of Formula (II) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl; additionally, said radical may be further optionally substituted with one to three substituents independently selected from the group consisting of C₁₋₆alkanyl and oxo;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano:

n is an integer from 1 to 3; and

enantiomers, diastereomers, tautomers, solvates, or pharmaceutically acceptable salts thereof.

Still another embodiment of the present invention is directed to uses of compositions comprising a compound of Formula (II) for treatment of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;

R⁴ is hydrogen;

n is an integer from 1 to 3; and

enantiomers, diastereomers, tautomers, solvates, or pharmaceutically acceptable salts thereof.

Still yet another embodiment of the present invention is directed to uses of compositions comprising a compound of Formula (II) for treatment of one or more of chronic pain (e.g., chronic neuropathic pain), acute neuropathic pain, inflammation (e.g., local inflammation or systemic inflammation), an inflammatory response, or an inflammatory condition, wherein:

R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆ alkanyl and oxo;

R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;

n is 1; and

enantiomers, diastereomers, tautomers, solvates, or pharmaceutically acceptable salts thereof.

Another embodiment of the present invention is directed to uses of compositions comprising a compound of Formula (I), Formula (II), or a combination thereof, for treatment of one or more of chronic pain (e.g., chronic neuropathic pain), acute neuropathic pain, inflammation (e.g., local inflammation or systemic inflammation), an inflammatory response, or an inflammatory condition, wherein said compound is selected from the group consisting of:
Pyridine-2-carboxylic acid (4-hydroxy-phenyl)-amide;
(S)-Pyrrolidine-2-carboxylic acid (4-hydroxy-phenyl)-amide;
N-(4-Hydroxy-phenyl)-2-mercapto-acetamide;
N-(4-Hydroxy-phenyl)-2-methylsulfanyl-acetamide;
N-(4-Hydroxy-phenyl)-2-methanesulfonyl-acetamide;
(R)-Pyrrolidine-2-carboxylic acid (4-hydroxy-phenyl)-amide;
1H-Pyrrole-2-carboxylic acid (4-hydroxy-phenyl)-amide;
1H-Indazole-3-carboxylic acid (4-hydroxy-phenyl)-amide;
5-Methyl-1H-pyrazole-3-carboxylic acid (4-hydroxy-phenyl)-amide;
3H-Imidazole-4-carboxylic acid (4-hydroxy-phenyl)-amide;
   pharmaceutically acceptable salts thereof; and
   combinations thereof.

In one embodiment, the instant disclosure is directed to a method of treating one or more of chronic pain (e.g., chronic neuropathic pain), acute neuropathic pain, inflammation (e.g., local inflammation or systemic inflammation), an inflammatory response, or an inflammatory condition, in a patient in need thereof by administering a therapeutically effective amount of 5-Methyl-1H-pyrazole-3-carboxylic acid (4-hydroxy-phenyl)-amide or pharmaceutically acceptable salt thereof to the patient.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 300 mg once a day to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 500 mg once a day to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 750 mg once a day to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 1000 mg once a day to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 2000 mg once a day to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 300 mg twice a day to a patient in need thereof in order to treat, prevent, or one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 500 mg twice a day to a patient in need thereof in order to treat, prevent, or one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 750 mg twice a day to a patient in need thereof in order to treat, prevent, or one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 1000 mg twice a day to a patient in need thereof in order to treat, prevent, or one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be orally administered in the methods of the present invention in an amount of about 2000 mg twice a day to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be topically administered in the methods of the present invention in an amount of about 1000 mg to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be topically administered in the methods of the present invention in an amount of about 2000 mg to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be topically administered in the methods of the present invention in an amount of about 3000 mg to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be topically administered in the methods of the present invention in an amount of about 4000 mg to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

In one embodiment, 5-Methyl-1H-pyrazole-3-carboxylic acid (4-hydroxy-phenyl)-amide or pharmaceutically acceptable salt thereof can be topically administered in the methods of the present invention in an amount of about 5000 mg to a patient in need thereof in order to treat, prevent, or ameliorate one or more of chronic pain (e.g., chronic neuropathic pain), acute neuropathic pain, inflammation (e.g., local inflammation or systemic inflammation), an inflammatory response, or an inflammatory condition.

The compounds of Formula (I) and (I) can be prepared in accordance with U.S. Pat. No. 7,718,692 which is hereby incorporated by reference.

### Additional Agents

In certain embodiments, the hetero-substituted acetanilide compound may be administered in combination with an additional active agent. Additional active agents may include, without limitations, a non-steroidal anti-inflammatory agent, acetaminophen, an opioid analgesic, a triptan, an anti-epileptic, a chemotherapeutic drug, or a combination thereof.

In certain embodiments, the combination of the hetero-substituted acetanilide compound with the additional active agent have an additive effect. The term “additive effect,” as used herein, refer to the overall consequence of the hetero-substituted acetanilide compound and the additional active agent being the sum of effects of the hetero-substituted acetanilide compound and the additional active agent acting independently.

In certain embodiments, the combination of the hetero-substituted acetanilide compound with the additional active agent have a synergistic effect. The term “synergistic effect,” as used herein, refer to the overall consequence of the hetero-substituted acetanilide compound and the additional active agent being enhanced as compared to the sum of effects of the hetero-substituted acetanilide compound and the additional active agent acting independently.

In certain embodiments, the hetero-substituted acetanilide compound and the additional active agent can be administered at the same time. The agents can be combined in the same dosage form or be in separate dosage forms. When in separate dosage forms the agents can be administered by the same route of administration (e.g., oral) or by different routes of administration (e.g., topical and oral). When in the same dosage form, hetero-substituted acetanilide compound and the additional active agent can be further formulated so as to independently release each of the active ingredients at a predetermined rate. Thus, for example, the pharmaceutical composition can be formulated as a coated formulation in which an active ingredient in the coating is released, followed by a release of the active ingredient in the core and vice versa.

In certain embodiments, the hetero-substituted acetanilide compound and the additional active agent can be administered simultaneously or sequentially. In certain embodiments, the agents are administered sequentially such that there is an overlap of the therapeutic interval provided by each agent. With sequential administration, the agents are in separate dosage forms and can be administered by the same route of administration (e.g., oral) or by different routes of administration (e.g., topical and oral).

The term “simultaneously” as used herein means that a dose of one agent is administered at the same time as another agent, regardless of whether the agents are administered separately via the same or different routes of administration or in a single pharmaceutical composition or dosage form. For example, a dose of acetaminophen may be administered at the same time as a dose of a compound of Formula (I) and/or (II).

The term “sequentially” as used herein means that a dose of one agent is administered first and thereafter a dose of another agent is administered second. For example, a dose of a acetaminophen may be administered and thereafter a dose of a compound of Formula (I) and/or (II) may be administered. The subsequent administration of the other agent may be inside or outside the dosing interval of agent that was administered first.

Exemplary non-steroidal anti-inflammatory agents include, without limitations, e.g., a COX-1 inhibitor, a COX-2 inhibitor or a non-specific COX inhibitor. In certain embodiments, the non-steroidal anti-inflammatory agent is aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid tolfenamic acid, diflurisal, flufenisal, piroxicam, sudoxicam, isoxicam or pharmaceutically acceptable salts thereof.

In other embodiments, the non-steroidal anti-inflammatory agent is rofecoxib, celecoxib, DUP-697, flosulide, meloxicam, 6-MNA, L-745337, nabumetone, nimesulide, NS-398, SC-5766, T-614, L-768277, GR-253035, JTE-522, RS-57067-000, SC-58125, SC-078, PD-138387, NS-398, flosulide. D-1367, SC-5766, PD-164387, etoricoxib, valdecoxib, parecoxib or pharmaceutically acceptable salts thereof.

Exemplary opioid analgesics can be, without limitations, e.g., alfentanil, buprenorphine, butorphanol, codeine, diamorphine, dextromoramide, dezocine, dihydrocodeine, fentanyl, hydrocodone, hydromorphone, levorphanol, meperidine, meptazinol, methadone, morphine, nalbuphine, nalorphine, opium, oxycodone, oxymorphone, pentazocine, propoxyphene, remifentanyl, sufentanyl, tapentadol, tramadol or pharmaceutically acceptable salts thereof.

Exemplary triptan can be, without limitations. e.g., sunatriptan, nzatriptan, naratriptan, eletriptan, donitriptan, almotriptan, frovatriptan, alvitriptan, zolmatriptan or pharmaceutically acceptable salts thereof.

Exemplary anti-epileptic can be, without limitations, e.g., gabapentin, pregabalin or pharmaceutically acceptable salts thereof.

Additional exemplary active agents can be, without limitations, lidocaine, menthol, or pharmaceutically acceptable salts thereof.

Examplary types of chemotherapeutic drugs include, without limitations, podophyllotoxins, taxanes, platinum complexes, vinca alkaloids, proteasome inhibitors, colchicines, eribulin, lenolidamide, ixabepilone, interpherons, thalidomide, etoposide, ifosfamide, procarbazine, cytarabine, gemcitabine, and arsenic. Example chemotherapeutic drugs include, but are not limited to, one or more of the following: anti-cancer alkylating or intercalating agents (e.g., Mechlorethamine, Chlorambucil, Cyclophosphamide. Melphalan, and Ifosfamide); antimetabolites (e.g., Methotrexate); purine antagonists and pyrimidine antagonists (e.g., 6-Mercaptopurine, 5-Fluorouracil, Cytarablle, Capecitabine and Gemcitabine); spindle poisons (e.g., Vinblastine, Vincristine, Vinorelbine and Paclitaxel); podophyllotoxins (e.g., Etoposide, Irinotecan, Topotecan); antibiotics (e.g., Doxorubicin, Bleomycin and Mitomycin); nitrosoureas (e.g., Carmustine, Lomustine); inorganic ions (e.g., Cisplatin, Carboplatin, Oxaliplatin or Oxiplatin); enzymes (e.g., Asparaginase); hormones (e.g., Tamoxifen, Leuprolide, Flutamide and Megestrol); proteasome inhibitors (such as Velcade); other kinase inhibitors (e.g., inhibitors of Src, BRC/Abl, kdr, flt3, aurora-2, glycogen synthase kinase 3 (“GSK-3”), EGF-R kinase (e.g., Iressa. Tarceva. VEGF-R kinase, PDGF-R kinase); antibodies, soluble receptor or other receptor antagonists against a receptor or hormone implicated in a cancer (including receptors such as EGFR, ErbB2, VEGFR, PDGFR, and IGF-R), and agents such as Herceptin (or other anti-Her2 antibodies). Avastin, and Erbitux. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In certain embodiments the weight ratio (w/w) of i) the additional active agent and ii) the compound selected from Formula (I) and/or (II), per unit dose, may range, e.g., from about 100:1 to about 1:100, from about 80:1 to about 1:80, from about 50:1 to about 1:50, from about 30:1 to about 1:30, from about 15:1 to about 1:15, from about 10:1 to about 1:10, from about 8:1 to about 1:8, from about 5:1 to about 1:5, from about 3:1 to about 1:3, or from about 2:1 to about 1:2. The disclosed weight ratios can be when the agents are combined in the same dosage form or when administered in separate dosage forms.

In certain embodiments of the present invention, i) the additional active agent and ii) the compound selected from Formula (I) and/or (II), is in an additive or synergistic amount with respect to its effect on one or more of chronic pain (e.g., chronic neuropathic pain), acute neuropathic pain, inflammation (e.g., local inflammation or systemic inflammation), an inflammatory response, or an inflammatory condition. In certain embodiments, the additional active agent is administered in a sub-therapeutic amount with respect to its effect on one or more of chronic pain (e.g., chronic neuropathic pain), acute neuropathic pain, inflammation (e.g., local inflammation or systemic inflammation), an inflammatory response, or an inflammatory condition if administered alone.

### Pharmaceutical Compositions

Even though the hetero-substituted acetanilide compounds of the present invention (including their pharmaceutically, acceptable salts and pharmaceutically acceptable solvates) can be administered alone, they will generally be administered in admixture with a pharmaceutical carrier, excipient or diluent selected with regard to the intended route of administration and standard pharmaceutical or veterinary practice. Thus, the present invention in certain embodiments is directed to pharmaceutical compositions and to a method for treating one or more of chronic pain (e.g., chronic neuropathic pain), acute neuropathic pain, inflammation (e.g., local inflammation or systemic inflammation), an inflammatory response, or an inflammatory condition, which method includes administering to a patient in need thereof a pharmaceutical and veterinary compositions comprising i) a hetero-substituted acetanilide compound, ii) optionally an additional active agent as described above, and iii) one or more pharmaceutically acceptable carriers, excipients or diluents.

Pharmaceutical compositions used in the present invention may be manufactured, e.g., by means of mixing, dissolving, granulating, emulsifying, encapsulating, entrapping or lyophilizing processes.

The pharmaceutically acceptable carriers, excipients or diluents used may be dependent upon the route of administration chosen.

For injection (e.g., for parenteral administration by bolus injection or continuous infusion), the hetero-substituted acetanilide compound (and optional additional active agent) may be formulated in aqueous solutions (e.g., in ampoules or in multi-dose containers), preferably in physiologically compatible buffers such as Hank's solution, Ringer's solution, or physiological saline buffer with or without organic solvents such as propylene glycol, polyethylene glycol and with or without preservatives.

For transmucosal administration, a penetrants may be used.

For oral administration, the hetero-substituted acetanilide compound (and optional additional active agent) can be formulated by combining the active agent(s) with pharmaceutically acceptable carriers. Such carriers enable the compound(s) to be formulated as tablets, pills, dragees, capsules, liquids, gels, syrups, slurries, suspensions, sublingual and buccal formulations and the like, for oral administration or ingestion by a patient. Suitable excipients are, in particular, fillers such as sugars, including lactose, sucrose, mannitol, or sorbitol; cellulose preparations such as, for example, maize starch, wheat starch, rice starch, potato starch, sodium starch glycollate, gelatin, gum tragacanth, methyl cellulose, hydroxypropylmethylcellulose, microcrystalline cellulose, sodium carbomethylcellulose and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions can also be in the form of capsules made of gelatin as well as soft, sealed capsules made of gelatin and a plasticizer, such as glycerol or sorbitol. The capsules may contain the active agents in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For administration by inhalation, the hetero-substituted acetanilide compound (and optional additional active agent) may be in the form of an aerosol to be administered from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be controlled with a valve used to deliver a metered amount. Capsules and cartridges of, e.g., gelatin for use in an inhaler or insufflator may be formulated containing a powder mix of the compound and a suitable powder base such as lactose or starch.

The hetero-substituted acetanilide compound (and optional additional active agent) may be formulated for parenteral administration, e.g., by bolus injection or continuous infusion. Formulations for injection may be presented in unit dosage form. e.g., in ampoules or in multidose containers with optionally, an added preservative. The compositions may be suspensions, solutions or emulsions in oily or aqueous vehicles, and may contain formulatory agents such as suspending, stabilizing and/or dispersing agents. In certain embodiments, the parenteral formulation can be a concentrate or a ready-to-use formulation.

Pharmaceutical compositions for parenteral administration include aqueous solutions of the hetero-substituted acetanilide compound (and optional additional active agent) in water-soluble form. Additionally, suspensions of the hetero-substituted acetanilide compound (and optional additional active agent) may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the conjugates to allow for the preparation of highly concentrated solutions.

Alternatively, the hetero-substituted acetanilide compound (and optional additional active agent) may be in powder form for constitution with a suitable vehicle, e.g., sterile, pyrogen-free water, before use.

The hetero-substituted acetanilide compound (and optional additional active agent) of the present invention may also be formulated in rectal compositions such as suppositories or retention enemas, using, e.g., conventional suppository bases such as cocoa butter or other glycerides.

Any of the pharmaceutical compositions of the present invention may, if desired, be presented in a pack or dispenser device, such as a FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Pharmaceutical compositions may be prepared, placed in an appropriate container, and labeled for treatment of an indicated condition, as set forth herein.

Thus, according to an embodiment of this aspect of the present invention, the pharmaceutical composition is packaged in a packaging material and identified in print, in or on the packaging material, for use in the treatment of pain and/or fever.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

The following examples and/or studies are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

### Example 1—Evaluation of Compound 1 in a Rat Formalin Test

5-Methyl-1H-pyrazole-3-carboxylic acid (4-hydroxy-phenyl)-amide (Compound 1) was tested in the rat formalin (1/%) model. The rat formalin test is a preclinical model of human painful conditions.

The formalin test in rat (or mice) is a valid and reliable model of nociception (sensation of pain) and is sensitive for various classes of analgesic drugs. Formalin is a solution of formaldehyde in water. The subcutaneous injection of dilute formalin induces pain in humans and nociceptive (presumptive of pain) behaviors in animals. Since it is painful when it is injected in humans, it is used as an animal model that is considered translational to human pain. This model is often used to screen novel compounds by recording formalin-induced nociceptive behaviors followed by scoring and analysis of the raw data.

### Formalin-Induced Pain Model

Hind-paw injection of formalin is a model used to assess intense persistent pain and evaluate analgesic drugs in laboratory animals. In this widely used model, a solution of formaldehyde is injected subcutaneously into mouse or rat paw. The effect of the formalin is indicated by the animal licking the injected paw, and shaking. This occurs principally in two phases. The early phase (Phase 1) is relatively shorter lasting (about 10 min), followed by a brief period of relative quiescence (about 5 min), after which the late phase (Phase II) responses occur over additional 50 minutes or so. Phase I is thought to result from direct activation of primary afferent sensory neurons (nociceptive pain). Phase 11 has been proposed to reflect the combined effects of both afferent input plus central sensitization in the dorsal horn of the spinal cord and brain, which is more characteristic of persistent or chronic pain.

In the formalin model, pain response scoring is determined by recording the pain-like behaviors per unit of time including licking, flinching, or raising or shaking of the injected paw. Analgesics such as morphine, gabapentin, and NMDA antagonists inhibit Phase II responses.

### Test System

Species: Rat

### Strain: Sprague Dawley rat

### Source: Charles River Kingston, USA

Age/weight at Arrival: 7-8 weeks, 175-225 g
Number of animals: 56 males including 2 spares

### Justification of Test System and Animal Use

The rat has been characterized in the literature as a suitable species for assessing test item effects on the rat carrageenan model of inflammation and pain. The research does not unnecessarily duplicate previous animal experiments. The research was conducted in the absence of acceptable non-animal alternatives to provide meaningful data and was designed such that it did not require an unnecessary number of animals to accomplish its objectives.

Only male animals were used for this model based on literature reports and to maintain consistency with previous historical data for this model.

### Animal Identification

Each animal was uniquely identified using indelible ink or tail-tattoo.

### Environmental Acclimation

A minimum acclimation period of 5 days was allowed between animal receipt and the start of carrageenan injection in order to accustom the animals to the laboratory environment.

### Selection, Assignment, and Replacement of Animals

Before dosing, animals were randomized based on body weight.

Before the initiation of dosing, any assigned animals considered unsuitable for use in the study were replaced by alternate animals obtained from the same shipment and maintained under the same environmental conditions.

After initiation of dosing, assigned animals that became unsuitable, that die or are euthanized during the replacement period could be replaced with alternate animals at the discretion of the Study Director.

### Animal Disposition

The disposition of all animals was documented in the study records. All animals remaining unassigned to groups after the last dosing occasion were released from the study and their disposition documented.

### Husbandry

### Housing

Animals were group-housed (up to 3 per bin) in solid-bottom caging with com-cob bedding (unless deemed inappropriate by the Study Director and/or Clinical Veterinarian) equipped with an automatic watering valve. Each cage was clearly labeled with a color-coded cage card indicating study, group, animal numbers and sex. Cages were arranged on the racks in group order.

### Environmental Conditions

The targeted conditions for animal room environment were as follows:

### Temperature: 19° C. to 25° C.

### Humidity: 30% to 70%

Light Cycle: 12 hours light and 12 hours dark (except during designated procedures)

### Food

PMI Nutrition International Certified Rodent Chow No. 5CR4 (14% protein) was provided ad libitum throughout the study, except during designated procedures. The same diet in meal form could be provided to individual animals as warranted by clinical signs (e.g., broken/damaged incisors or other health changes). The feed was analyzed by the supplier for nutritional components and environmental contaminants. Results of the analysis were provided by the supplier and are on file at the Test Facility.

It is considered that there are no known contaminants in the feed that would interfere with the objectives of the study.

### Water

Municipal tap water which has been softened, purified by reverse osmosis and exposed to ultraviolet light was freely available (except during designated procedures). Water bottles could be provided, if required.

Periodic analysis of the water was subcontracted to management authorized analytical laboratories. The analytical results were retained in the archives of CR-SEN.

It is considered that there were no known contaminants in the water that could interfere with the objectives of the study.

### Animal Enrichment

For psychological/environmental enrichment, animals were provided with items such as chewing object, hiding tunnel and nesting material except during designated activities.

### Veterinary Care

Veterinary care was available throughout the course of the study and animals were examined by the veterinary staff as warranted by clinical signs or other changes. All veterinary examinations and recommended therapeutic treatments were documented in the study records.

In the event that animals showed signs of illness or distress, the responsible veterinarian could make initial recommendations about treatment of the animal(s) and/or alteration of study procedures, which had to be approved by the Study Director or scientific designate. All such actions were properly documented in the study records and, when appropriate, by study plan amendment. Treatment of the animal(s) for minor injuries or ailments could be approved without prior consultation with the Sponsor representative when such treatment would not impact fulfillment of the study objectives. If the condition of the animal(s) warranted significant therapeutic intervention or alterations in study procedures, the Sponsor representative was contacted, when possible, to discuss appropriate action. If the condition of the animal(s) was such that emergency measures had to be taken, the Study Director and/or attending veterinarian attempted to consult with the Sponsor representative prior to responding to the medical crisis, but the Study Director and/or veterinarian had authority to act immediately at his/her discretion to alleviate suffering. The Sponsor representative was fully informed of any such events.

### Experimental Design

| | | | TI 1 Dose vol, | |
|---|---|---|---|---|
| | | TI 1 Dose | pretreatment | |
| Groups* | Group treatment | (mg/kg) | and route | N |
| 1 | 1% Formalin/Vehicle | 0 | 10 mL/kg, 1 hr, PO | 9 |
| | (0.5% HPMC) | | | |
| 2 | 1% Formalin/Compound 1 | 300 | 10 mL/kg, 1 hr, PO | 9 |
| 6 | 1% Formalin/Tramadol | 50 | 10 mL/kg, 1 hr, PO | 9 |

*All animals from each Group received a 50 μl injection of 1% formalin solution intra-plantarly (plantar subcutaneous) into the left hind paw.
PO: Oral gavage administration; IP: Intra-peritoneal injection.
TI: test item.

### Dosing, Formalin Injection and Video Recording and Scoring

Before the formalin test, animals were randomized into treatment groups based on body weight. Before injection of formalin, the animals were administered with vehicle, Compound 1 and Tramadol as depicted in the table above.

For all Groups, animals were acclimated to the observation chamber for about 15 minutes immediately prior to formalin injection. All animals received an intraplantar subcutaneous injection of 50 μL of 1% formalin into the left hind paw and were then be placed in the observation chamber immediately and formalin-evoked spontaneous nociceptive behaviors in the rats was continuously recorded for 0-60 min using a commercial camcorder. Camera was turned on at least 5 min before formalin injection and verified for proper functioning.

Scoring from the recorded video files was performed off-line using a PC by an observer who has been validated to score such nociceptive behavior in rodents. The total time spent in a 5 min bin was recorded using a stop-watch for the following nociceptive behavior: flinching, shaking, biting and licking of the injected paw. Effects of the drugs was assessed in the following bins: 0-5 minutes from early phase (phase-1) and 20-35 minutes from late phase (phase-II).

In certain cases, direct observation and scoring of formalin-evoked nociceptive behavior without video recording could be performed by an observer if required. The observer who was doing off-line video scoring or direct observational scoring of the nociceptive behavioral responses in the rats was blinded to dosing groups to ensure that interpretation of behavioral assessment is not biased.

### Justification of Route and Dosage Levels

The dose levels and routes of administration selected for this study were based on Sponsor's information from in-vitro and PK analysis data and were not expected to cause any acute toxicity in the animals.

Intra-paw injection of formalin is a commonly used model to measure spontaneous nociceptive behaviors in rodents (Dubuisson, D. and Dennis, S. G. Pain 4:161, 1977). Dose levels of Tramadol. Duloxetine and Gabapentin were selected based on previous CRL studies (5900104, 590766).

### Inducing Agent, Test Items And Vehicle

### Inducing Agent

Identity:Formalin solution, neutral buffered 10%

Storage Conditions:Kept in a refrigerator set to maintain 4° C.

### Vehicle for Formalin

Identity: Phosphate buffered saline (PBS)

Storage Conditions:Kept in a refrigerator set to maintain 4° C.

### Positive Control

Identity: Tramadol

### Vehicle for Tramadol

Identity: Phosphate buffered saline, PBS (1×, pH 7.4)

Storage Conditions:Controlled room temperature

### Test Item (TI) 1

Identity: 5-Methyl-1H-pyrazole-3-carboxylic acid (4-hydroxy-phenyl)-amide (Compound 1)

### Vehicle for Test Item

Identity:0.5% Hydroxy-propyl methyl cellulose (medium viscosity) in ultrapure water

### Dose Formulation and Analysis

### Preparation of Test Items (Ti)

Test Item 1 formulations were prepared by suspending in the vehicle 0.5% HPMC in UPW.

### Preparation of Formalin

A 1% formalin solution was freshly prepared by dilution of the 10% stock of formalin at 9:1 in phosphate buffered saline (PBS, 1X, pH 7.4) on the day of study. The formulation was maintained at room temperature until use.

### Preparation of Positive Controls

Tramadol was prepared on the day before dosing in the respective vehicles, stored at RF and dispensed at room temperature.

### Other In-Life Procedures, Observations, and Measurements

The in-life procedures, observations, and measurements listed below were performed for all animals, including spare animals. During the study, additional evaluations to those described below and/or scheduled, and considered necessary by the Study Director and/or Veterinarian to assess health status were conducted and duly documented. More frequent observations could be undertaken if considered appropriate.

### Mortality/Moribundity Checks

Frequency:Once daily
Procedure:Cageside observation.

### Clinical Observations

Frequency:On the day of dosing
Procedure:Cageside observation.

### Body Weights

Frequency:Day of dosing
Procedure: Animals will be individually weighed.

### Terminal Procedures

Animals euthanized for humane reasons as well as all animal from the study (after video recording) were euthanized by deep isoflurane anesthesia and exsanguination of the abdominal aorta and were discarded without further examination.

The carcass of animals found dead during the conduct of the study was discarded without further examination.

### SUMMARY

Widely used, the formalin test is an animal model of continuous pain resulting from formalin(formaldehyde)-induced tissue injury. In this model, spontaneous pain-related responses can be observed in a freely moving unrestrained animal. Behaviors can be scored over a prolonged period such that the onset and duration of analgesia (pain relief) can be assessed. It is a particularly useful model for assessing analgesic efficacy, because it encompasses inflammatory, neurogenic, and central mechanisms of nociception.

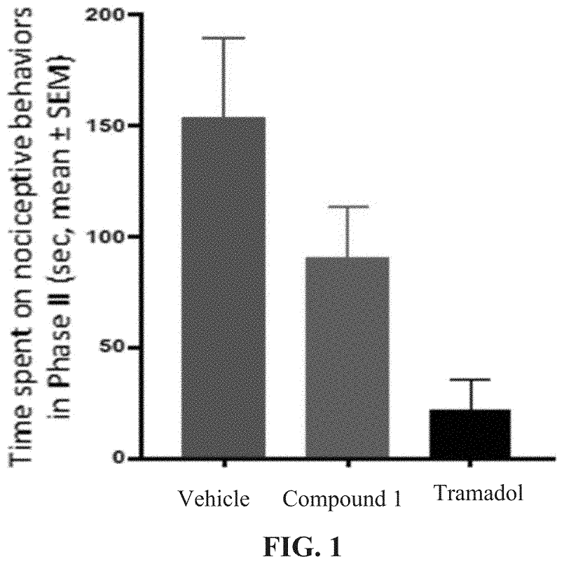

Compound 1 was tested in rat formalin (1%) test by Charles River Laboratories. Tramadol was used as a positive control. As can be seen in FIG. 1. Compound 1 (300 mg/kg, oral) inhibited nociceptive behaviors (was antinociceptive) in Phase II of the rat formalin test. Compound 1 is a non-opioid and a non-NSAID drug. The results of the rat formalin test implied that Compound 1 would display analgesic efficacy in persistent human pain conditions involving nociceptive, inflammatory, neurogenic, and central mechanisms.

### Example 2—Evaluation of Compound 1 in a Carrageenan Paw-Edema Model

5-Methyl-1H-pyrazole-3-carboxylic acid (4-hydroxy-phenyl)-amide (Compound 1) was tested in the rat carrageenan paw-edema model. The rat carrageenan model is a preclinical model of inflammation and anti-inflammatory efficacy.

### Carrageenan Paw-Edema Model

The acute inflammatory response is well-known to be characterized by an increase in vascular permeability, an increase in blood flow, as well as infiltration of neutrophils and macrophages. Moreover, an edema (swelling caused by excess fluid trapped in tissues) is often formed due to exudation of fluid and of plasma proteins and accumulation of leukocytes at the inflammatory site. To date, several experimental models of paw edema have been described by investigators. As one of the best-characterized acute inflammatory models, carrageenan-induced mouse/rat model has been increasingly used to test new anti-inflammatory drugs as well as to study the mechanisms involved in inflammation.

Inflammation models induced by carrageenan are frequently-used acute inflammation models because they are well-researched and because they exhibit a high degree of reproducibility. Carrageenan, which is a natural linear sulfated polysaccharide that is extracted from red edible seaweeds, stimulates the release of inflammatory and proinflammatory mediators, including bradykinin, histamine, tachykinins, reactive oxygen, and nitrogen species. The typical signs of inflammation following the intraplantar (into a paw) injection of carrageenan include edema, hyperalgesia, and erythema.

Generally, animals are injected with carrageenan in one hind footpad, usually, half an hour or an hour after they are treated with the test compound while the other footpad is injected with saline as a control. The measurement of paw volume/thickness is then performed at specified time points.

### Objective

The objective of this study was to evaluate the effect of 5-Methyl-1H-pyrazole-3-carboxylic acid (4-hydroxy-phenyl)-amide (Compound 1) on paw edema in rat induced by intra-plantar injection of λ-Carrageenan into the left hind paw.

### Induction Agent, TEST Items and Positive Control

### Induction Agent

Identity: λ Carrageenan

Storage Conditions:Controlled room temperature

Supplier: Sigma-Aldrich

### Vehicle for Carrageenan

Identity:0.9% Sodium chloride, USP (saline)

Storage Conditions:Controlled room temperature

### Test Item Identification

Identity:5-Methyl-1H-pyrazole-3-carboxylic acid (4-hydroxy-phenyl)-amide (Compound 1)

### Vehicle for Test Item

Identity:0.5% Hydroxy-propyl methyl cellulose (medium viscosity) in ultrapure water

### Dose Formulation Preparation

### Preparation of Carrageenan

Carrageenan was dissolved in physiological saline (0.9% sodium chloride, USP) to obtain concentrations of 1% (1 g/100 mL (w/v)). The formulation was prepared on the day before injection and kept over-night on a stir plate at room temperature. The carrageenan solution was vortexed/stirred before each injection.

### Preparation of Test Item

Test Item formulations were prepared by suspending in the vehicle 0.5% HPMC in UPW.

### Preparation of TritonX-100

Triton solution was prepared as required by addition of 15 drops of Triton X-100 in sodium chloride solution (1 g/L). Preparation was stored at room temperature.

### Test System

Species: Rat

### Strain: Crl:CD (SD) Sprague Dawley rat

### Sex: Male

### Source: Charles River Kingston, USA

Age/weight at Arrival: 7-8 weeks, 175-225 g
Number of animals: 32 males including 2 spares

### Justification of Test System and Animal Use

The rat has been characterized in the literature as a suitable species for assessing test item effects on the rat carrageenan model of inflammation and pain. The research does not unnecessarily duplicate previous animal experiments. The research was conducted in the absence of acceptable non-animal alternatives to provide meaningful data and was designed such that it did not require an unnecessary number of animals to accomplish its objectives.

Only male animals were used for this model based on literature reports and to maintain consistency with previous historical data for this model.

### Animal Identification

Each animal was uniquely identified using indelible ink or tail-tattoo.

### Environmental Acclimation

A minimum acclimation period of 5 days was allowed between animal receipt and the start of carrageenan injection in order to accustom the animals to the laboratory environment.

### Selection, Assignment, and Replacement of Animals

Before the initiation of dosing, any assigned animals considered unsuitable for use in the study were replaced by alternate animals obtained from the same shipment and maintained under the same environmental conditions.

Animals were assigned into treatment groups based on body weight and baseline paw volume data.

After initiation of dosing, assigned animals that became unsuitable, that die or are euthanized during the replacement period could be replaced with alternate animals at the discretion of the Study Director.

### Animal Disposition

The disposition of all animals was documented in the study records. All animals remaining unassigned to groups after the last dosing occasion were released from the study and their disposition documented.

### Husbandry

### Housing

Animals were group-housed (up to 3 per bin) in solid-bottom caging with corn-cob bedding (unless deemed inappropriate by the Study Director and/or Clinical Veterinarian) equipped with an automatic watering valve. Each cage was clearly labeled with a color-coded cage card indicating study, group, animal numbers and sex. Cages were arranged on the racks in group order.

### Environmental Conditions

The targeted conditions for animal room environment were as follows:

### Temperature: 19° C. to 25° C.

### Humidity: 30% to 70%

Light Cycle: 12 hours light and 12 hours dark (except during designated procedures)

### Food

PMI Nutrition International Certified Rodent Chow No. 5CR4 (14% protein) was provided ad libitum throughout the study, except during designated procedures. The same diet in meal form could be provided to individual animals as warranted by clinical signs (e.g., broken/damaged incisors or other health changes). The feed was analyzed by the supplier for nutritional components and environmental contaminants. Results of the analysis were provided by the supplier and are on file at the Test Facility.

It is considered that there are no known contaminants in the feed that would interfere with the objectives of the study.

### Water

Municipal tap water which has been softened, purified by reverse osmosis and exposed to ultraviolet light was freely available (except during designated procedures). Water bottles could be provided, if required.

Periodic analysis of the water was subcontracted to management authorized analytical laboratories. The analytical results were retained in the archives of CR-SEN.

It is considered that there were no known contaminants in the water that could interfere with the objectives of the study.

### Animal Enrichment

For psychological/environmental enrichment, animals were provided with items such as chewing object, hiding tunnel and nesting material except during designated activities.

### Veterinary Care

Veterinary care was available throughout the course of the study and animals were examined by the veterinary staff as warranted by clinical signs or other changes. All veterinary examinations and recommended therapeutic treatments were documented in the study records.

In the event that animals showed signs of illness or distress, the responsible veterinarian could make initial recommendations about treatment of the animal(s) and/or alteration of study procedures, which had to be approved by the Study Director or scientific designate. All such actions were properly documented in the study records and, when appropriate, by study plan amendment. Treatment of the animal(s) for minor injuries or ailments could be approved without prior consultation with the Sponsor representative when such treatment would not impact fulfillment of the study objectives. If the condition of the animal(s) warranted significant therapeutic intervention or alterations in study procedures, the Sponsor representative was contacted, when possible, to discuss appropriate action. If the condition of the animal(s) was such that emergency measures had to be taken, the Study Director and/or attending veterinarian attempted to consult with the Sponsor representative prior to responding to the medical crisis, but the Study Director and/or veterinarian had authority to act immediately at his/her discretion to alleviate suffering. The Sponsor representative was fully informed of any such events.

### Experimental Design

| | | TI Dose | Dose | Dose formulation | |
|---|---|---|---|---|---|
| | | level | volume | concentration | |
| Gr. | Group/treatment | (mg/kg) | (mL/kg) | (mg/mL) | N |
| 1 | Cg/Vehicle | 0 | 10 | 0 | 10 |
| 2 | Cg/Test Item | 300 | 10 | 30 | 10 |
| | dose 1 | | | | |
| 3 | Cg/Test Item | 600 | 10 | 60 | 10 |
| | dose 2 | | | | |

Cg: Carrageenan; TI: Test Item (Test item will dosed at 1 hour before Carrageenan injection)

### Model Induction

On Day 1, carrageenan (1%; 100 uL) was injected subcutaneously into the plantar aspect of the left hindpaw of rats from all groups. Carrageenan injection was performed in conscious rats by restraining with hand and injection was done in the middle of the plantar surface area. The carrageenan solution was vortexed/stirred before each injection.

### Test Item and Vehicle Control Administration

Route of Administration Oral gavage (PO)
Dosing Regimen: Single dose on Day 1, 1 h before Carrageenan injection.
Method: The day of dosing was designated as Day 1. The animals were not sedated. The volume for each dose was administered over one injection based on the body weight.

### Justification of Route and Dosage Levels

The dose levels and routes of administration selected were determined and targeted to a therapeutic range and have been used in other longer-term animal studies at the same dose without toxicity.

Dose level for diclofenac was selected based on previous internal validation studies 1602 and literature reports (Nagkura Y et al. 2003. Allodynia and hyperalgesia in adjuvant-induced arthritic rats: time course of progression and efficacy of analgesics. J Pharmacol Exp Ther. 2003 August; 306(2):490-7).

The rat carrageenan edema model is a commonly used preclinical model to assess the anti-inflammatory effects of test compounds in vivo. (Morris. CJ. Carrageenan-induced paw edema in the rat and mouse. Methods Mol Biol. 2003:225:115-21).

### In-Life Procedures, Observations, and Measurements

The in-life procedures, observations, and measurements listed below will be were performed for all main study animals, including spare animals, where appropriate. During the study, additional evaluations to those described below and/or scheduled and considered necessary by the Study Director and/or Veterinarian to assess health status were conducted and duly documented. More frequent observations could be undertaken if considered appropriate.

### Mortality/Moribundity Checks

Frequency: Once daily
Procedure: Cageside observation.

### Body Weights

Frequency:Pretreatment and day of dosing.
Procedure. Animals will be individually weighed.

### Paw Volume Measurements

Paw volume (left hind paw) was measured before carrageenan injection (baseline) and then at 1, 3, 6 and 8 hours (+/−2 minutes) after carrageenan injection (on Day 1).

Measurements will be done using a digital plethysmometer as below:

In brief, the Plethysmometer is a volume meter, designed for accurate measurements of inflammation-induced swelling. It consists of a water filled cell into which the paw along with the ankle joint is dipped. A transducer records the differences in water level caused by volume displacement and provides LCD readout of the exact volume gain due to swelling.

It is very important that each animal, and each paw, is measured at the exact same point.

Limb volumes were recorded onto a data sheet as they were measured. One trial per animal were recorded for each time point. A second trial could be performed when confirmation of value was required due to animal's movement.

### Summary

Widely-used, the carrageenan paw-edema test is an animal model of inflammation. In this model, swelling of the paw occurs in response to inflammatory processes and the release of chemical inflammatory mediators. The effect can be measured as an increase in paw volume secondary to increased fluid in the paw (edema). It is a particularly good model for assessing anti-inflammatory efficacy of substances.

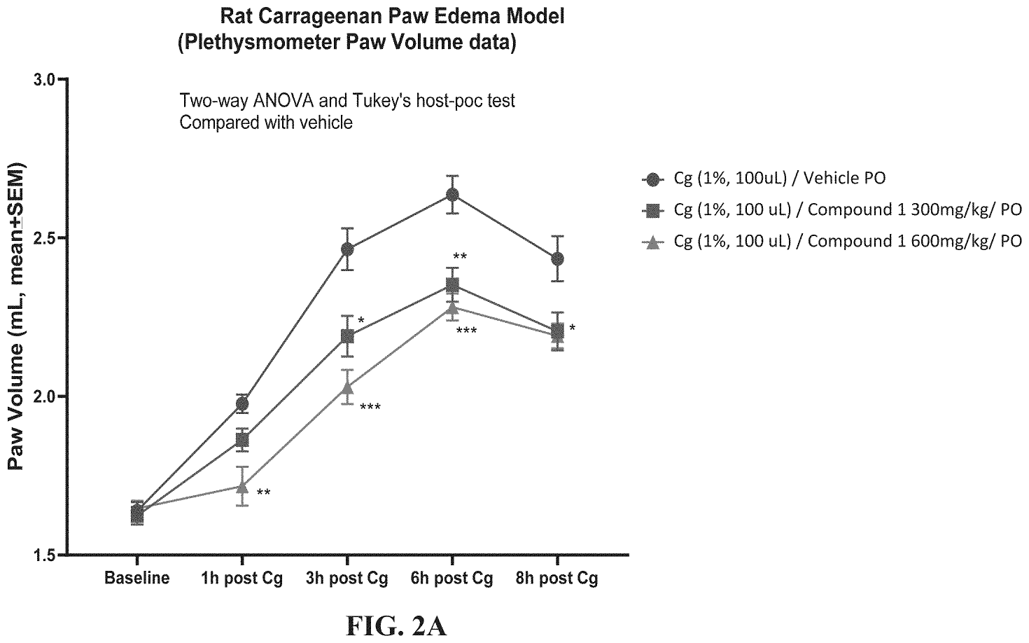

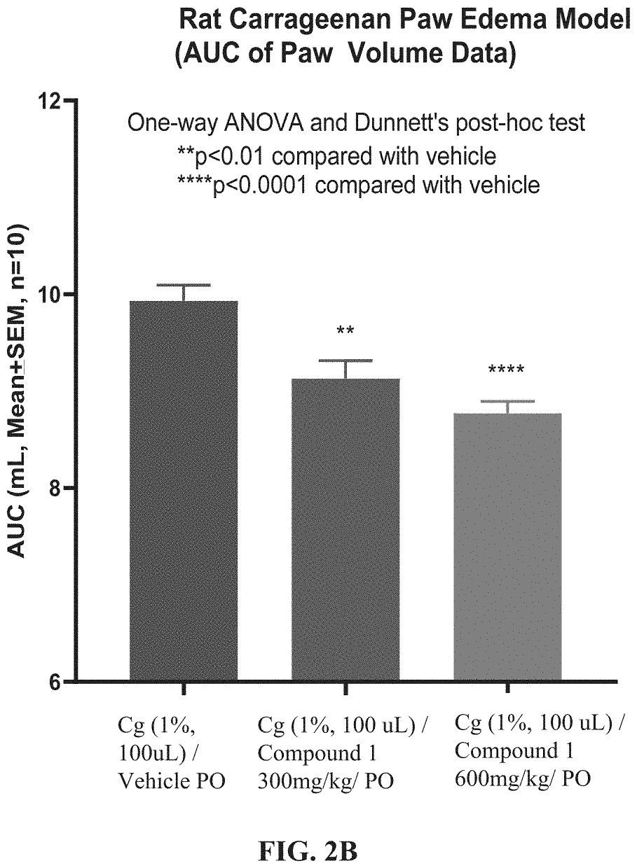

Compound 1 was tested in the rat carrageenan-induced paw-edema model at Charles River Laboratories. As can be seen in FIG. 2A, Compound 1 (300 mg/kg and 600 mg/kg, orally) dose-relatedly inhibited carrageenan-induced paw-edema in rats. The duration of the effect of a single oral dose of Compound 1 was longer than 8 hours (i.e., illustrating a long lasting anti-inflammatory effect after a single dose, which may be, without being construed as limiting, suggestive of a physiological change such as modulation of an adenosine A3 receptor).

FIG. 2B shows the area under the curve (AUC) of the Paw Volume Data illustrated in FIG. 2A. FIG. 2B further confirmed that there was a statistically significant difference in the AUC results that were exhibited by the 300 mg/kg dose of Compound 1 as compared to the 600 mg/kg dose of Compound 1.

This is strong evidence that Compound 1 has anti-inflammatory activity, and would be expected to provide anti-inflammatory efficacy in human conditions that have an inflammatory component.

### Example 3

A single-center, randomized, double-blind, placebo- and active-controlled, parallel group study was performed to evaluate the efficacy, safety and pharmacokinetic profile of a single dose of 5-Methyl-1H-pyrazole-3-carboxylic acid (4-hydroxy-phenyl)-amide (Compound 1), over a 24-hour on-site period in moderate to severe pain after third molar extractions in up to 280 male subjects was performed.

Healthy male subjects, ages 18 to 45 years, were screened by medical history, vital signs, electrocardiogram (ECG), and clinical laboratory tests. Eligible subjects returned to the clinic on the day of surgery and completed baseline vital signs and clinical laboratory tests.

The subjects were randomized such that one group received placebo (Group 1), one group received 1000 mg of Compound 1 (Group 2), and one group received 1000 mg acetaminophen (Group 3). All Groups received the treatment dose within up to 4.5 hours after oral surgery (third molar extraction).

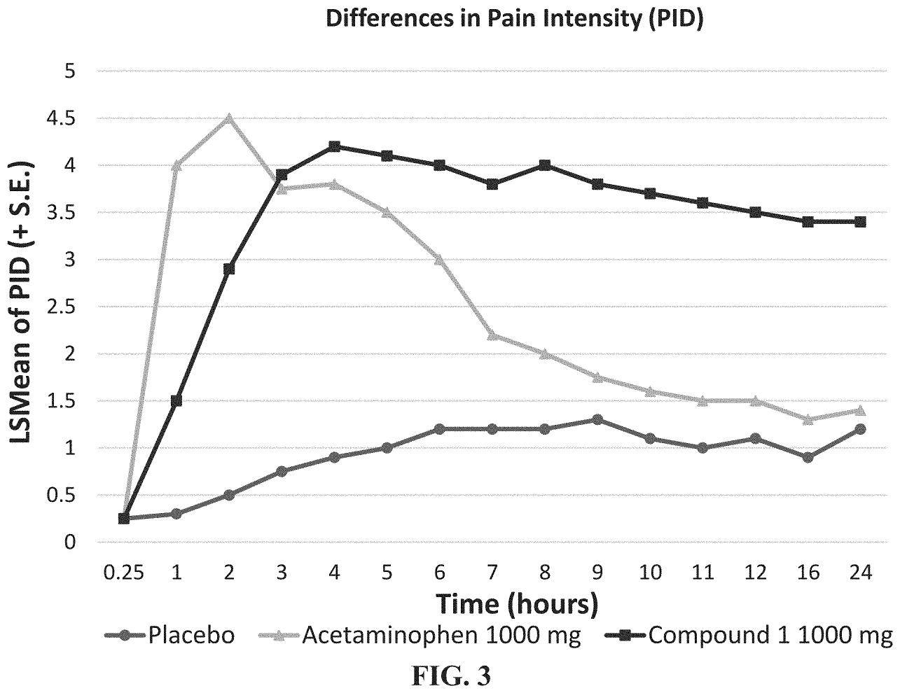

Pain intensity/pain relief assessments were collected at 0.25, 0.5, 0.75, 1, 1.5, 2, hourly up to 12, 16, and 24 hours post-dose and at time of first rescue medication (oral ibuprofen). Pain intensity difference from baseline (PID) scores at each time point are shown in FIG. 3. As can be seen from FIG. 3, subjects treated with 1000 mg of Compound 1 (Group 2) had a significantly greater and longer lasting PID score compared with subjects treated with 1000 mg of acetaminophen (Group 3). This result (i.e., long lasting PID score) is surprising as the T_{1/2} for Compound 1 was about 10 hours (i.e., the approximate time for the plasma concentration of Compound 1 to reduce to half of its initial value was about 10 hours). This unexpected result makes the compounds of the present invention suitable for chronic pain and/or inflammatory indications as disclosed herein.

Without being construed as limiting, it is believed that this evidences Compound 1's effectiveness (or analgesic potential) in treatment and/or prevention and/or amelioration of chronic pain and/or neuropathic pain (in addition to acute pain).

### Prophetic Example 4

5-Methyl-1H-pyrazole-3-carboxylic acid (4-hydroxy-phenyl)-amide (Compound 1) treatment is tested in the suppression of the development of mechanical allodynia after severe spinal cord contusion injury in the rat.

### Surgical Methods

Thirteen week-old female Lewis rats (Charles River, Sulzfeld Germany) are housed under a 12:12 h dark/light regime and allowed free access to water and food. After one week of habituation the animals undergo general anesthesia with a mixture of isoflurane and air (induction: 5% isoflurane, maintenance: 2.2% isofluorane). A Th10 laminectomy is performed without rupturing the dura and a severe contusive SCI (severe spinal cord injury) (25 gcm NYU/MASCIS II impactor) {Gruner, 1992 #3} was induced. After suturing muscle and skin, a subcutaneous (s.c.) injection of an ml of Ringers Lactate is given. Bladders are emptied manually 2 times a day until spontaneous voiding returned (usually within 1 week). The lesion severity is verified by the impact velocity and contusion depth of the impactor rod. Animals with an impact velocity error>5% are excluded from further analysis. After injury, individual rats are randomly assigned into a treatment group. The following groups were used:
Group I: SCI model+vehicle (1.5% Carboxymethylcellulose (CMC) in sterile water) by gavage for seven days
Group 2: SCI model+Compound 1 (10 mg/kg/day) in vehicle by gavage for 7 days

### Assessment of Mechanical Sensitivity

The mechanical sensitivity response is measured as the direct pressure stimulus required for eliciting foot withdrawal in nonrestrained conditions. The person performing the behavioral tests is blinded to the experimental groups. Animals are habituated to the testing apparatus for at least 20 min before testing. Each animal is subjected to the stimulation of a series of von Frey filaments ranging from 0.4 to 15 g (log force 3.61, 3.84, 4.08, 4.31, 4.56, 4.74, 4.93 and 5.18) using the Up-Down paradigm according to Chaplan (Chaplan et al., 1994). The selected von Frey filament is pressed against the plantar surface of the hind paw to the point of 30 bending for 3 s. Paw withdrawal response is considered as the positive response. The 50% threshold force needed for paw withdrawal is calculated for both hind paws of each rat and the mean value of both hind paws is used to represent the mechanical sensitivity of this animal.

### Prophetic Results

The mechanical sensitivity (indicated by the 50% threshold force for paw withdrawals) is determined by the Up-Down method using von Frey filaments.

All rats are baseline tested before surgery and tested again on day 28 post surgery, because this is the first time point at which all rats can sit with the hind paws in plantar position. At baseline all animals reach the maximal 50% threshold force of 15 g, as consequence there were no differences between the two groups. It is expected that the mechanical hypersensitivity is reduced in all Compound 1 treated dosage groups.

### Prophetic Example 5

Subjects are diagnosed with neuropathic pain (chronic and ongoing for more than 3 months in contrast to an acute injury of nerve or nervous system).

In case of low back pain (mixed pain) a possible, probable or definite neuropathic pain component can be diagnosed via screening tools at screening/baseline visits, e.g. PAIN DETECT, LANSS, DN4).

A 12 week study is designed to compare 5-Methyl-1H-pyrazole-3-carboxylic acid (4-hydroxy-phenyl)-amide (Compound 1) to placebo and to a standard (pregabalin).

There are 2-3 dosing arms for Compound 1 (including 1000 and 2000 mg/day).

After comparison of efficacy endpoints, it is demonstrated that Compound 1 shows improvement vs. placebo and improvement or comparable effects vs. standard in pain intensity and signs and symptoms of neuropathy, hyperexcitability after measurement by e.g. NPSI (neuropathic pain symptom inventory. PAINDETECT for LBP) or other validated tools.

### Prophetic Example 6

A patient is treated for chronic lower back pain with an oral dosage form comprising between 500 mg and 2000 mg of 5-Methyl-1H-pyrazole-3-carboxylic acid (4-hydroxy-phenyl)-amide or a pharmaceutically acceptable salt thereof.

### Prophetic Example 7

A patient is treated for chronic bladder pain with an oral dosage form (e.g., a tablet or capsule) comprising between 500 mg and 2000 mg of 5-Methyl-1H-pyrazole-3-carboxylic acid (4-hydroxy-phenyl)-amide or a pharmaceutically acceptable salt thereof.

A number of embodiments of the invention have been described. Nevertheless, it will be understood that various modifications may be made without departing from the spirit and scope of the invention. Accordingly, other embodiments are within the scope of the following claims. It should be apparent, however, to those skilled in the art that many more modifications besides those already described are possible without departing from the inventive concepts herein. The inventive subject matter, therefore, is not to be restricted except in the spirit of the disclosure.

All patents, patent publications and publications mentioned herein are incorporated herein by reference in their entirety to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

1. A method of treating chronic pain comprising administering to a patient in need thereof a therapeutically effective amount of a hetero-substituted acetanilide compound.

2. The method of claim 1, wherein the chronic pain is associated with inflammation.

3. The method of claim 1, wherein the compound is selected from Formula (I): 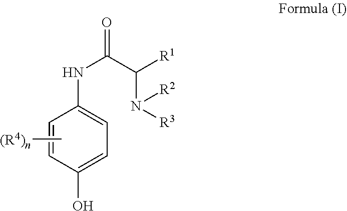
wherein:
R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆alkanyl and oxo;
R³ is hydrogen or C₁₋₆alkanyl;
R⁴ is a substituent selected from the group consisting of hydrogen, C₁₋₆alkanyl, C₁₋₆ alkanyloxy, fluorinated alkanyl, fluorinated alkanyloxy, halogen, hydroxyl, nitro, amino, C₁₋₆ alkanylamino; C₁₋₆dialkanylamino and cyano;
n is an integer from 1 to 3; and
enantiomers, diastereomers, tautomers, solvates, or pharmaceutically acceptable salts thereof, or from Formula (II) 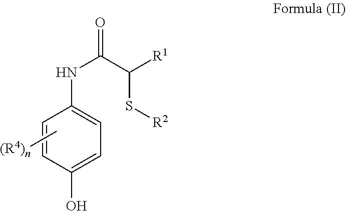
wherein:
R¹ is a substituent independently selected from the group consisting of hydrogen and C₁₋₆ alkanyl;
R² is a substituent independently selected from the group consisting of hydrogen, C₁₋₆ alkanyl and dioxo;
or R¹ and R² taken together with the atoms to which they are attached form a radical selected from the group consisting of a 5 to 10 membered cycloheteroalkanyl, 5 to 10 membered cycloheteroalkenyl and a 5 to 10 membered heteroaryl, wherein, in addition to the heteroatom N, said radical may optionally contain 1 to 3 additional heteroatoms, independently selected from the group consisting of O, N and S; additionally, said radical may be further optionally substituted with one to three substituents independently selected from the group consisting of C₁₋₆ alkanyl and oxo;
R⁴ is a substituent selected from the group consisting of hydrogen, C₁₋₆alkanyl, C₁₋₆alkanyloxy, fluorinated alkanyl, fluorinated alkanyloxy, halogen, hydroxyl, nitro, amino, C₁₋₆ alkanylamino; C₁₋₆dialkanylamino and cyano;
n is an integer from 1 to 3; and
enantiomers, diastereomers, tautomers, solvates, or pharmaceutically acceptable salts thereof, or a combination thereof.

4. The method of claim 3, wherein the hetero-substituted acetanilide compound modulates the adenosine A3 receptor.

5. The method of claim 1, comprising administering a compound of Formula (I).

6. The method of claim 1, comprising administering a compound of Formula (II).

7. The method of claim 1, comprising administering a compound selected from the group consisting of:
Pyridine-2-carboxylic acid (4-hydroxy-phenyl)-amide;
(S)-Pyrrolidine-2-carboxylic acid (4-hydroxy-phenyl)-amide;
N-(4-Hydroxy-phenyl)-2-mercapto-acetamide;
N-(4-Hydroxy-phenyl)-2-methylsulfanyl-acetamide;
N-(4-Hydroxy-phenyl)-2-methanesulfonyl-acetamide;
(R)-Pyrrolidine-2-carboxylic acid (4-hydroxy-phenyl)-amide;
1H-Pyrrole-2-carboxylic acid (4-hydroxy-phenyl)-amide;
1H-Indazole-3-carboxylic acid (4-hydroxy-phenyl)-amide;
5-Methyl-1H-pyrazole-3-carboxylic acid (4-hydroxy-phenyl)-amide;
3H-Imidazole-4-carboxylic acid (4-hydroxy-phenyl)-amide;
pharmaceutically acceptable salts thereof; and
combinations thereof.

8. The method of claim 1, comprising administering 5-Methyl-1H-pyrazole-3-carboxylic acid (4-hydroxy-phenyl)-amide or pharmaceutically acceptable salt thereof.

9. The method of claim 1, wherein the administration is by a route comprising oral, intravenous, nasal, inhalational, topical, buccal, rectal, pleural, peritoneal, vaginal, intramuscular, subcutaneous, transdermal, epidural, intratrachael, otic, intraocular, or intrathecal.

10. The method of claim 1, wherein the chronic pain comprises neuropathic pain, nociceptive pain, non-nociceptive pain, or a combination thereof.

11. The method of claim 1, further comprising administering a compound comprising a non-steroidal anti-inflammatory agent, acetaminophen, an opioid analgesic, a triptan, an anti-epileptic, menthol, lidocaine, or a combination thereof.

12. The method of claim 11, wherein the combination has an additive effect.

13. The method of claim 11, wherein the combination has a synergistic effect.

14. The method of claim 1, wherein the administration of the hetero-substituted acetanilide compound is by an oral route.

15. The method claim 1, wherein the therapeutically effective amount of the hetero-substituted acetanilide compound ranges from about 200 mg to about 2000 mg.

16. The method of claim 15, wherein the administration is once daily or twice daily.

17. The method of claim 1, wherein the administration of the hetero-substituted acetanilide compound is by a topical route.

18. The method of claim 17, wherein the hetero-substituted acetanilide compound is formulated as a gel, a cream, or a transdermal patch.

19. The method of claim 17, wherein the therapeutically effective amount of the hetero-substituted acetanilide compound ranges from about 500 mg to about 5000 mg.

20. The method of claim 17, wherein the hetero-substituted acetanilide compound is formulated in combination with menthol or lidocaine.

21-27. (canceled)
